(19) United States
(12) Patent Application Publication
(10) Pub. No.: US 2007/0082393 A1
(43) Pub. Date: Apr. 12, 2007

(54) Polymer coated nanofibrillar structures and methods for cell maintenance and differentiation

(76) Inventors: Muhammad Lodhi, Eagan, MN (US); Tahmina Naqvi, Blaine, MN (US); Gary Opperman, St. Louis Park, MN (US); Hoo Young Chung, Bloomington, MN (US); Melvin S. Schindler, Piscataway, NJ (US)

Correspondence Address: KAGAN BINDER, PLLC, SUITE 200, MAPLE ISLAND BUILDING, 221 MAIN STREET NORTH, STILLWATER, MN, 55082, US

(21) Appl. No.: 11/489,964

(22) Filed: Jul. 20, 2006

Related U.S. Application Data
(60) Provisional application No. 60/700,860, filed on Jul. 20, 2005.
(60) Provisional application No. 60/719,351, filed on Sep. 22, 2005.
(60) Provisional application No. 60/764,849, filed on Feb. 3, 2006.

(51) Int. Cl. C12N 5/06; C12N 5/08; C12M 1/12

(52) U.S. Cl. 435/325; 435/368; 977/902; 435/348; 435/297.1

(57) ABSTRACT

The invention provides cell-adherent polymeric coatings for articles having a nanofibrillar structure. The coatings include a synthetic, non-biodegradable polymer having at least one pendent amine group, wherein the polymer is covalently immobilized on the article via latent reactive groups. The invention also provides methods for the long term culturing of cells using the polymer coated nanofibrillar structures. The polymer coated nanofibrillar structures of the invention have been found to be particularly useful for the growth and differentiation of cells, including neural precursors.

### CROSS-REFERENCE TO RELATED APPLICATIONS

The present non-provisional Application claims the benefit of U.S. Provisional Application having Ser. No. 60/700,860, filed on Jul. 20, 2005, and entitled, Polymeric Coatings and Methods for Cell Maintenance and Differentiation; U.S. Provisional Application having Ser. No. 60/719,351, filed on Sep. 22, 2005, and entitled, Polymer Coated Nanofibrillar Structures and Methods for Cell Maintenance and Differentiation; and U.S. Provisional Application having Ser. No. 60/764,849, filed on Feb. 3, 2006, and entitled, Polymer Coated Nanofibrillar Structures and Methods for Cell Maintenance and Differentiation.

### FIELD OF THE INVENTION

The invention relates to nanofibrillar structures having coatings that include a non-biodegradable amine- presenting polymer and methods for promoting the adherence of cells on surfaces that include these coatings. The invention also relates to methods of differentiating cells, as well as methods for maintaining cells on surfaces having these coatings.

### BACKGROUND OF THE INVENTION

Various approaches have been used to provide surfaces that are suitable for cell attachment and growth. Many cells are anchorage dependent, meaning that they must demonstrate some type of attachment to a substrate in order to proliferate or differentiate. In vivo, cells can attach to protein factors present in the basement membrane, which is a structure that supports an overlying epithelium or endothelium. The basement membrane consists of a membrane called the basal lamina and an underlying network of collagen fibrils. Due to its capability to provide an excellent substrate for cell attachment and growth, artificial surfaces for cell attachment based on components found in basement membranes have been fabricated. Such artificial surfaces have been used in vivo, such as in implantable medical devices, and in vitro, such as in cell culture articles.

Charged surfaces have been used to promote the attachment of cells to a substratum. However, not all charged surfaces are suitable for the sufficient attachment of cells during culturing processes. For example, some negatively charged surfaces do not provide a suitable substrate because many cells do not display a sufficient amount of positively charged proteins to mediate cell attachment to the surfaces.

Natural polypeptide-based cell attachment factors such as collagen, fibronectin, and laminin have been used to enhance the attachment of cells to a substrate. Biodegradable synthetic polymeric cations such as polylysine and polyornithine have also been used to provide coatings that promote the attachment of various anchorage dependent cell types. One problem with the use of these types of polymeric materials is that they can degrade over a period of time by proteases which may become present in the liquid medium of a culture, or that are present in vivo in serum Therefore, surfaces containing these materials may only be useful for cell attachment for a limited period of time. Properties related to cell adhesion may be compromised by the degradation of the materials present within the coating.

Furthermore, some coatings that are used to promote cell attachment may also be problematic from the standpoint that coating materials may be lost from the coating if not properly attached to the surface of the article. These materials may then become present in the liquid media or body fluid and affect cells that come in contact with it. For example, polymeric materials lost from the coating may bind the surface of the cells and affect cell attachment to the substrate or may affect cell-cell interactions in culture. Some polymeric materials may also be detrimental to cell viability.

Therefore, there is a need to provide improved polymeric coatings for articles used in processes involving culturing cells. Such improved coatings could provide for useful cell culture articles that would benefit processes involving the maintenance and differentiation of primary cells, cells lines that are difficult to culture, and stem cells. Such coatings could also be used to coat the surfaces of implantable medical articles for in vivo use. Accordingly, this would benefit the technology of tissue specific regeneration for the treatment of a wide array of diseases and conditions.

### SUMMARY OF THE INVENTION

In one aspect, the present invention provides coatings for cell culture articles that are useful in methods for culturing cells, and, in particular, methods for keeping cells in culture for a protracted period of time (long term cultures). Culturing, as used herein, refers to processes involving placing metabolically active cells in a cell culture article that includes a nanofibrillar structure with a polymeric coating. The polymer-coated nanofibrillar structure of the present invention has been shown to provide an ideal surface for long-term cell culture because of the stability of the polymeric coating, the excellent surface for cell attachment, and the broad applicability for culturing a wide variety of cell types.

A nanofibrillar structure refers to a mesh-like network of nanofibers, which are fiber structures that have an average diameter, of about 1000 nm or less, such as in the range of about 1 nm to about 1000 nm, and more preferably in the range of about 50 nm to about 1000 nm. In some aspects, the nanofibrillar structure is formed from by a process that includes the electrospinning of a polymer solution. In some aspects the nanofibrillar structure includes a polyamide, a polyester, a similar synthetic polymer, or combinations thereof. In some aspects the polyamide is selected from a nylon polymer. In yet other aspects the nanofibers are formed by a process that includes the crosslinking of water- or alcohol-soluble nylon polymers to provide water- or alcohol-insoluble nanofibers.

In many aspects, the nanofibrillar structure is used in conjunction with another cell culture article. The nanofibrillar structure can be adapted or configured for placement on a cell culture surface, such as the surface of a cell culture vessel. Examples of cell culture vessels include multi-well plates, dishes, and flasks. Therefore, a nanofibrillar structure can be obtained in a shape suitable for placement into a culture vessel, such as a cell culture well, and a cell culturing process can be performed in the cell culture well with the nanofibrillar structure on a surface (such as at the bottom) of the well.

Cells can be cultured on the polymer-coated nanofibrillar structure, in a liquid medium to provide a desired metabolically active cellular state. The polymer-coated nanofibrillar structure can be used to promote one or more metabolically active cellular states, including states wherein the cell is quiescent (a non-proliferative and non-differentiating state), states of cell proliferation, and states of cell differentiation.

In another aspect of the invention, the coatings are provided on an implantable medical device comprising a nanofibrillar structure. In some ways, similar to the function of the coatings as provided in vitro, the coatings can be used on these surfaces to promote cell attachment. This is useful for a number of applications, including promotion of tissue formation, epithelialization, and angiogenesis.

The inventive polymer-coated nanofibrillar structure includes a non-biodegradable polymer having pendent amine groups, wherein the coating also includes one or more latent reactive groups. In the coating, at least a portion of the latent reactive groups are reacted to covalently bind the polymer to the surface of the nanofibrillar structure.

In the coating, the latent reactive groups can be provided on the polymer as pendent latent reactive groups. Alternatively, pendent latent reactive groups can be included on a compound, such as a crosslinking agent, independent of the polymer and then used to couple the polymer to the surface of the nanofibrillar structure.

Latent reactive groups refer to groups that respond to specific applied external stimuli to undergo active specie generation with resultant covalent bonding to a target. The latent reactive groups generate active species such as free radicals, nitrenes, carbenes, and excited states of ketones upon absorption of external electromagnetic or kinetic (thermal) energy. In one aspect, the latent reactive group is a photoreactive group that can be activated to an active state to provide bonding between the polymer and the surface of a cell culture article. Exemplary photoreactive groups include aryl ketones, such as acetophenone, benzophenone, anthraquinone, anthrone, and anthrone-like heterocycles (for example, heterocyclic analogs of anthrone such as those having nitrogen, oxygen, or sulfur in the 10-position), or their substituted (for example, ring substituted) derivatives.

It is believed that the binding via the pendent latent reactive groups provides a coating wherein the polymer is optimally configured on the nanofibrillar surface to promote cell adhesion

The polymeric coating on the nanofibers provides amine groups that promote cell attachment to the nanofibrillar structure. In some aspects of the invention, the polymer of the present invention includes a pendent amine-containing group of the following formula:
—R₁R₂NR₃R₄

wherein R₁ is: 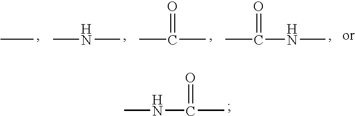
wherein R₂ is C₁-C₈ linear or branched alkyl; and

wherein R₃ and R₄ are both attached to the nitrogen and are individually H or C₁-C₆ linear or branched alkyl.

In some aspects, R₁ is 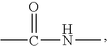
R₂ is C₂-C₄ linear or branched alkyl; and R₃ and R₄ are both attached to the nitrogen and are individually H, CH₃, or C₂H₅.

Exemplary amine containing groups include those found on polymerizable monomers such as 3-aminopropylmethacrylamide (APMA), 3-aminoethylmethacrylamide (APMA), and dimethylaminopropylmethacrylamide (DMAPMA). In some aspects, polymers including pendent amine groups and latent reactive groups can be formed by copolymerizing a monomer having a group —R₁R₂NR₃R₄ as defined above with a comonomer bearing a latent reactive group. In other aspects, a polymer can be formed by polymerizing a monomer having the formula R₁R₂NR₃R₄ and then reacting one or more pendent amine groups with a compound having a latent reactive group.

In other aspects, the polymer having pendent amine groups is selected from polyethyleneimine (PEI), polypropyleneimine (PPI), and polyamidoamine. PEI can be formed by the polymerization of ethylene imine; optionally a monomer having a polymerizable group and a latent reactive group can be copolymerized with ethylene imine to form PEI having pendent latent reactive groups. In one specific aspect the polymer includes polyethyleneimine with one or more latent photoreactive group(s).

A coating including a polymer having at least one pendent amine and at least one latent reactive group can be formed on the surface of the nanofibrillar structure using any suitable method. One method for forming the coating includes disposing a pre-formed polymer on the nanofibrillar surface and then activating the latent reactive groups to bind the polymer to the surface. In some aspects the latent reactive groups can be pendent from the polymer. In other aspects the latent reactive groups can be independent of the polymer. Alternatively monomeric material can be polymerized on the nanofibrillar surface to form a polymer having pendent amine and one or more latent reactive groups that couple the polymer to the surface.

The polymeric coatings of the present invention have been shown to be useful for the preparation of cell culture articles that include nanofibrillar structures. The nanofibrillar structure can be associated with cell culture articles including multi-well cell culture plates, cell culture dishes, cell culture bags, cell culture tubes, microcarriers, and cell culture bottles.

The nanofibrillar structure can be fabricated to have dimensions that are optimal for one or more aspects of the cell culturing process, as based on, for example, the particular cell type(s) being cultured, the number of cells being cultured, the length of culturing, and any optional cell culture apparatus used in conjunction with the coated nanofibrillar structure. In some aspects, the nanofabrillar structure has an area and depth suitable for use in conjunction with multi-well cell culture apparatus. For example, polymer coated nanofibrillar structure formed on a support can be configured to fit within a well of a multi-well culture apparatus. The depth of the nanofibrillar structure can vary, but it has been found that depths in the range of about 0.1 μm to 10 μm, and in the range of about 1 μm to about 5 μm can provide particularly effective substrates for cell culture.

The nanofibrillar structure can also be engineered to provide a network nanofibers having a desired spacing between the nanofibers, the spacing resembling pores in the structure. In many aspects, the nanofiber interlocking networks have relatively small spaces between the fibers such as of about 0.01 microns to about 25 microns, and in some cases about 0.2 microns to about 10 microns.

The coated surfaces of three-dimensional nanofiber-based cell culture articles can resemble scaffoldings on to which cells can attach. Depending on the type(s) of cells cultured on the nanofibrillar structure and the spacing between the nanofibers, the cells may be cultured in two dimensions or three dimensions. In a two dimensional culturing process, cells may attach to the coated nanofibers generally in one plane, whereas in a three dimensional culturing process, the cells may attach to the nanofiber in more than one plane, within the network of nanofibers.

The nanofibrillar structure can be provided in a form suitable for cell culturing processes. In some aspects the nanofibrillar structure is provided on a support. The support can be selected from glass substrates, such as glass microscope slides, glass cover slips, microspheres, and glass films; polymer substrates, such as polymer films; and other biomaterial substrates suitable for cell culturing. The support can have certain properties useful for the preparation and/or use of a cell culture device. In some specific aspects the support has one or more properties such as manipulability and/or flexibility, resistance to sterilization, resistance to degradation by radiation, chemical inertness, transparency, non-flammability, and smooth surface properties. In some specific aspects, the support includes a halogenated thermoplastic resin, such as halogenated fluorinated-chlorinated resins. Specifically, the support can include chlorotrifluoroethylene (CTFE). In other specific aspects, the support is about 0.25 mm or less.

A number of advantages for the preparation of cell culture articles and in methods for culturing cells have become apparent based on the inventive findings described herein.

First, the use of a coating including a polymer having pendent amine groups and latent reactive groups provides a remarkably effective and efficient way of providing an adherent surface (i.e., as relating to cellular adherence) to a nanofibrillar structure. In many cases, a liquid composition including the polymer can be applied to a nanofibrillar structure and then treated, thereby forming a coating of polymeric material on some or all of the nanofibers contacted by the liquid composition. In some cases a coating of polymeric material is formed on a portion of the nanofibrillar structure, such as a portion that is contacted by cells during the culturing process. This can provide an economic advantage, as additives to the material used to form the nanofibers are not necessarily required.

The coating compositions and methods described herein can also provide an adherent surface without compromising the topography of the overall structure provided by the nanofibers. It is thought that the methods of the present invention provide an extremely thin, yet very effective, polymeric coated layer on the surface of the nanofibers. That is, a polymeric coating is formed around individual nanofibers, rather than on the gross surface of the nanofibrillar structure. Traditional polymer coating processes (such as those where a polymer is dried down on a substrate) may result in webbing of the coating on the surface and change the topography of the nanofiber surface. However, the present polymeric coatings are very thin and proportional to the dimensions of the nanofibers of the nanofibrillar structure.

The nanofibrillar structure comprising a coating with a polymer having pendent amine groups is able to promote excellent adherence of cells during the culture process. This allows cells that display some degree of anchorage dependency to attach to the coating and exhibit one or more metabolic activities depending on the type of cell that is cultured and the type of media that the cell is cultured in. The polymer-coated nanofibrillar structures are therefore particularly useful for culturing cells that are non-adherent, poorly adherent, or moderately adherent. The inventive polymer-coated nanofibrillar structures are also particularly useful for providing coatings that allow cell proliferation and differentiation.

The invention provides polymer-coated nanofibrillar structures that are remarkably stable and effective for cell culturing processes. Accordingly, these structures of the present invention are particularly useful for procedures involving long term culturing of cells.

In these aspects, it has been found that the present polymer-coated nanofibrillar structures are particularly advantageous, as the amine-presenting polymer does not degrade in the presence of the culture medium and therefore can be used to promote the adherence of cells in culture for a considerable period of time. This is in comparison to coatings that are primarily composed of degradable natural polymers such as polypeptides and polysaccharides, as well as biodegradable synthetic polymers. These types of biodegradable coatings may degrade over a shorter period of time (such as a couple of weeks) and loose their ability to promote the adherence of cells in culture.

In addition, since the polymers of the present invention are covalently bonded to the surface of the nanofibers of the nanofibrillar structure, there is minimal or no loss of the amine-presenting polymer from the surface. This is advantageous in many regards; first, since over the culture period, the potential for the cells to become or remain attached to the nanofibrillar surface will not change. That is, the amount of amine-presenting polymer attached to the nanofibrillar surface will not significantly change over time. Second, there is minimal risk of loss of the amine-presenting polymer into the culture. This is also advantageous, because polymer lost in the culture may otherwise change the properties of the cells. For example, the cells may become non-adherent, or may lose other properties conveyed by proteins on the surface of the cells. In addition, in some cases, particular types of polymers lost from a coated surface into the media may have a toxic effect on the cells. For example, toxic effects have reported for some polyethyleneimines having a molecular weight of 25 kDa and greater in different culture systems when the PEI was added extracellularly (Fischer et al. (1998) Eur. J. Cell Biol. 48, 108; Fischer et al. (1999) Pharm. Res. 16, 1723-1729). Therefore, the polymer-coated nanofibrillar structures of the present invention overcome some of the shortcomings found in coated substrates that have been traditionally used to promote cell attachment and culturing in the prior art.

Although the present coatings on the polymer-coated nanofibrillar structures include a non-biodegradable amine-presenting polymer, other non-biodegradable or degradable materials, such as biodegradable polymers or bioactive molecules,.may be present in the coating. For example, while biodegradable polymers may be present in the coating and provide an advantage for culturing cells for a shorter time period, the non-biodegradable polymer remains present in the coating and provides an adherent surface during protracted periods of culturing.

In some aspects, the invention provides a method for culturing of cells on polymer-coated nanofibrillar structures. The method includes the step of obtaining a nanofibrillar structure, the nanofibrillar structure having a coating that includes a non-biodegradable synthetic polymer having at least one pendent amine groups and at least one latent reactive group that couples the polymer to the surface of the article. Cells are then placed in contact with the polymer-coated nanofibrillar structures, typically in an environment (liquid medium). The cells are able to adhere to the coating on the nanofibrillar cell culture article, so the cells can be maintained in a desired physiological state or be induced to have a desired physiological state.

In some aspects, the method includes culturing the cells in a liquid medium for a protracted period of time. A protracted period of time generally refers to a period of time that is greater than 14 days. When indicated, the protracted period of time may be greater than 21 days, greater than 28 days, greater than 35 days, greater than 42 days, greater than 49 days, or greater than 56 days. In some aspects, therefore, the cells also may be kept in culture for a time period in the range of about 14 to about 60 days. A distinct advantage of the invention is that the cells do not have to be transferred to a new nanofibrillar culture article having a fresh coating capable of promoting cell adherence. However, during the period of long term culturing, the liquid media can be changed, such as by replacement or by supplementation, to provide an environment that is suitable to achieve the desired physiological state.

In some aspects, over a period of the culturing process, the method can be used to maintain cells in a state of low metabolic activity (for example, maintaining quiescent cells). That is, in some aspects, cells can be maintained on the polymer-coated nanofibrillar structures in an appropriate media without promoting a metabolic change in the cells, such as one that may change the morphology of the cells. This method can also be useful for maintaining cells, and can include expanding the population of cells by cell proliferation. Exemplary cell types that can be maintained in cell culture using the polymer-coated nanofibrillar structures of the present invention include undifferentiated cells, such as stem cells, or partially or fully differentiated cell types, such hepatocytes, islet cells, neurons, and astrocytes. The undifferentiated cells can include multipotent, totipotent, or pluripotent cell types.

In this regard, the polymer-coated nanofibrillar structures are particularly useful in that they can be used for long-term maintenance of cells without a need to replace the coated article over a period of time.

In other aspects, over a period of the culturing process, the polymer-coated nanofibrillar structures can be used in methods to promote the differentiation of cells. Any suitable pre-differentiated or progenitor cell type can be used. The method can include the steps of obtaining a polymer-coated nanofibrillar structure and then disposing pre-differentiated cells on the structure, wherein the cells adhere to the coated structure. The method also includes a step of culturing the cells in the presence of an environment (liquid medium) that includes a component that can change the metabolic activity of the cells, leading to a change in one or more cellular aspects, such as cell morphology. The component can be a differentiation factor, which refers to any sort of component that promotes the maturation of the pre-differentiated cells into a partially or fully differentiated state. The method then includes the step of differentiating the cells that are in contact with the nanofibrillar coated surface. In some aspects, the cells are differentiated on the polymer-coated nanofibrillar structures for a period of time greater than 14 days, greater than 21 days, greater than 28 days, greater than 35 days, greater than 42 days, greater than 49 days, or greater than 56 days. In some aspects, the cells can be differentiated for a time period in the range of about 14 to about 60 days, depending on the initial seeding density of the cells. During the period of long term culturing, the liquid media can be changed, such as by replacement or by supplementation, to provide an environment that is suitable to achieve the desired physiological state.

Exemplary cell types that can be differentiated according to the present invention include primary cells, neural precursors, bone marrow cells, stem cells, such as embryonic (blastocyst derived) stem cells, and the like.

In some aspects, the method is used to promote the maturation of neural precursor cells into a desired differentiated neuronal cell type. The polymer-coated nanofibrillar structures of the present invention have been shown to promote the attachment of neural precursor cells, which can then be cultured for a protracted period of time in the presence of one or more desired differentiation factors. The polymer-coated nanofibrillar structures have been shown to promote neurite outgrowth and/or elongation, whereas neural precursors cultured on traditionally coated articles under the same media conditions did not survive. The polymer-coated nanofibrillar structures also promoted the appearance of mature neuronal markers in a subset of neuronal cells growing on the coated nanofibers. The present polymer-coated nanofibrillar structures have also been shown to promote the formation of neural precursors into astrocytes.

### BRIEF DESCRIPTION OF THE DRAWINGS

FIG. 1 is a graph showing the results of PC12 cell attachment on various photo-polymer coated and uncoated flat surfaces.

FIG. 2 is a graph showing the results of PC12 cell attachment on photo-polymer coated and uncoated nanofiber structures.

FIG. 3 are bright field microscopic images of PC12 cells growing on photo-poly(APMA)-coated nanofibers (3A) and uncoated nanofibers (3B).

FIG. 4 are fluorescence microscopic images of phalloidin stained PC12 cells having been grown on photo-poly(APMA)-coated nanofibers for a period of 24 hours.

FIG. 5 is a graph showing the results of HFF cell attachment on various photo-polymer coated and uncoated flat surfaces.

FIG. 6 are fluorescence microscopic images of BrdU incorporation in PC12 cells grown on photo-poly(APMA)-coated nanofibers (A) and uncoated nanofibers (B) and DAPI staining of these cells (A′) and (B′) respectively.

FIG. 7 are fluorescence microscopic images of β-III tubulin-stained PC12 cells grown on various photo-polymer coated and uncoated flat polystyrene surfaces.

FIG. 8 are fluorescence microscopic images of β-III tubulin-stained PC12 cells grown on various photo-polymer coated and uncoated flat nanofiber surfaces.

FIG. 9 are fluorescence microscopic images of β-III tubulin-stained PC12 cells grown on photo-poly(APMA)-coated nanofibers versus other commercially available cell substrates.

FIG. 10 is a fluorescence microscopic image of nestin/BrdU stained ES-D3 cells grown on photo-poly(APMA)-coated nanofibers.

FIG. 11 are fluorescence microscopic images demonstrating neurite morphology of beta III tubulin-stained ES-D3 grown on photo-poly(APMA)-coated nanofibers and uncoated nanofibers. The image shows that ES-D3 cells differentiate into process bearing neurons with longer neurite lengths compared to neurons growing on uncoated nanofibers where the processes are short and stubby.

FIG. 12 are fluorescence microscopic images of GFAP-stained ES-D3 grown on photo-poly(APMA)-coated nanofibers and uncoated nanofibers.

FIG. 13 is a fluorescence microscopic image demonstrating extensive neurite morphology of β-III tubulin-stained PC12 cells grown on photo-poly(APMA)-coated nanofibers and differentiated for a period of 30 days.

FIG. 14 is a fluorescence microscopic image demonstrating β-III tubulin-stained positive neurons (A) and Neurofilament-stained neurons, which are a subset of the β-III tubulin-stained positive neurons.

FIG. 15 is a fluorescence microscopic image showing GFAP⁺-stained Type I and Type II astrocytes.

### DETAILED DESCRIPTION

The embodiments of the present invention described below are not intended to be exhaustive or to limit the invention to the precise forms disclosed in the following detailed description. Rather, the embodiments are chosen and described so that others skilled in the art can appreciate and understand the principles and practices of the present invention.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

In some aspects, the present invention provides reagents and methods for providing a coating to the surface of nanofibers of a nanofibrillar structure, the coating including polymeric material having at least one pendent amine group and at least one latent reactive group, wherein at least one latent reactive group on the polymer is used to couple the polymer to the surface of the nanofibers of the nanofibrillar structure.

A “cell culture article” refers to any portion of a cell culture apparatus. For example, a cell culture article can be an article having a nanofibrillar structure. A cell culture article can also be a receptacle used in a cell culture process, such as a cell culture vessel. In some cases two or more cell culture articles (such as a nanofibrillar structure and a cell culture container) form a cell culture apparatus; in other cases a single cell culture article, such as the nanofibrillar structure, constitutes a cell culture apparatus.

A “nanofibrillar structure” refers to a mesh-like network of nanofibers. A nanofibrillar structure can be a cell culture article and can be included in any sort of cell culture apparatus wherein cell attachment is desired, or where a cell culture process is performed. In many cases an article that includes a network of nanofibers includes a network of nanofibers in addition to one or more other non-nanofiber materials. For example, a nanofibrillar structure can include a network of nanofibers on a support, wherein the support is fabricated from a material that is different than the nanofibers. A nanofibrillar structure can also be used with articles that are not used in in vitro cell culture processes.

The nanofibrillar structure can be placed on the surface of another cell culture article, such as a cell culture vessel. In many aspects, the nanofibrillar structure can be “adapted for insertion” into another article. This means that the nanofibrillar structure can be manufactured or fabricated for use in, or to the dimensions of one or more surfaces of another article, such as a cell culture vessel. The nanofibrillar structure can be sized for use in or to the dimensions of a surface of the culture vessel by, for example, cutting down the nanofibrillar structure to a particular size, for example cutting a piece of the nanofibrillar structure from an associated sheet, roll, or mat, to a size suitable for insertion onto a surface of the culture vessel.

A “cell culture vessel” is an example of a cell culture article and, as used herein, means a receptacle that can be associated with the nanofibrillar structure and can contain media for culturing a cell or tissue. The cell culture vessel may be glass or plastic. Preferably the plastic is non-cytotoxic. Exemplary cell culture vessels include, but are not limited to, single and multi-well plates, including 6 well and 12 well culture plates, and smaller welled culture plates such as 96, 384, and 1536 well plates, culture jars, culture dishes, petri dishes, culture flasks, culture plates, culture roller bottles, culture slides, including chambered and multi-chambered culture slides, culture tubes, coverslips, cups, spinner bottles, perfusion chambers, bioreactors, and fermenters.

A nanofibrillar structure can be in the form of a “mat” which as used herein means a densely interwoven, tangled, or adhered mass of nanofibers. The distribution of nanofibers in the mat may be random or oriented. A mat may be unwoven or net. A mat may or may not be deposited on a support. A mat has a thickness of about 100 nm to about 10,000 nm, or about 1000 nm to about 5000 nm.

The polymeric coatings of the present invention can be formed on any type of suitable nanofiber of a nanofibrillar structure. In some aspects, it is desirable to provide a cell culture article having nanofibrillar structure that provides a surface sufficient for the growth and differentiation of one or more cell types. Such an article ideally has a surface that supports the particular morphology of the differentiating cells. For example, in the case of differentiation of neural precursors, the nanofibrillar surface allows the formation of neurites or other features of neural cells which are greater than 10 μm, or greater than 200 μm.

Depending on the method of fabrication and the types of cells that are cultured, cells can grow in one plane, or more than one plane on the nanofibrillar structure. Generally, the coated surfaces of cell culture articles that include a nanofibrillar structure resemble scaffoldings on which cells can attach.

The polymeric coatings can be formed on the nanofibers of a nanofibrillar structure, wherein the nanofibers can be fabricated from a wide variety of materials. The materials used to form the nanofibrillar structure are referred to herein as “nanofiber materials” whereas the materials used to form the polymeric coatings on the nanofibers are herein referred to as “coating materials.”

Exemplary nanofibrillar structures are described in U.S. Patent Pub. No. 2005/0095695A1.

The nanofibrillar structure provides an environment for the culturing of metabolically active cells comprising one or more nanofibers, wherein the structure is defined by a network of one or more nanofibers. In some embodiments, the nanofibrillar structure comprises a substrate wherein the nanofibrillar structure is defined by a network of one or more nanofibers deposited on a surface of the substrate. The nanotopography, the topography of the nanofiber network and the arrangement of the nanofibers of the nanofiber network in space, of the nanofibrillar structure is engineered to provide an in vitro biomimetic substratum that is tissue compatible for the promotion of homotypic or heterotypic cell growth and/or cell differentiation in single layer or multi-layered cell culture. The nanofibrillar structures can be layered to form a multi-layered nanofibrillar assembly, cellular array, or tissue structure.

The term “network” as used herein means a random or oriented distribution of nanofibers in space that is controlled to form an interconnecting net with spacing between fibers selected to promote growth and culture stability. The network has small spaces between the fibers comprising the network forming pores or channels in the network. The pores or channels have a diameter of about 0.01 microns to about 25 microns, and more typically about 0.2 microns to about 10 microns, through a thickness. Advantageously, the polymeric coatings that are formed on the nanofibers do not significantly reduce the diameter of the pores or channels.

A network may comprise a single layer of nanofibers, a single layer formed by a continuous nanofiber, multiple layers of nanofibers, multiple layers formed by a continuous nanofiber, or mat. The network may be unwoven or net. A network may have a thickness of about the diameter of a single nanofiber to about 10 μm. Physical properties of the network including, but not limited to, texture, rugosity, adhesivity, porosity, solidity, elasticity, geometry, interconnectivity, surface to volume ratio, fiber diameter, fiber solubility/insolubility, hydrophilicity/hydrophobicity, fibril density, and fiber orientation may be engineered to desired parameters. Advantageously, the polymeric coating of the present invention be can formed on the nanofibers without significantly changing the beneficial properties provided by the network of nanofibers. For example, the polymeric coating does not alter the structural features of the nanofibrillar structure in such a way as that it reduces its function as a biomimetic substratum.

The term “nanofiber” as used herein means a polymer fine fiber comprising a diameter of about 1000 nanometers or less.

A wide range of polymeric materials can be used as nanofiber materials in the preparation of the nanofibrillar structures. Nanofiber materials can include both addition polymer and condensation polymer materials such as polyolefin, polyacetal, polyamide, polyester, cellulose ether and ester, polyalkylene sulfide, polyarylene oxide, polysulfone, modified polysulfone polymers and mixtures thereof. Exemplary materials within these generic classes include polyethylene, poly(ε-caprolactone), poly(lactate), poly(glycolate), polypropylene, poly(vinylchloride), polymethylmethacrylate (and other acrylic resins), polystyrene, and copolymers thereof (including ABA type block copolymers), poly(vinylidene fluoride), poly(vinylidene chloride), polyvinyl alcohol in various degrees of hydrolysis (87% to 99.5%) in crosslinked and non-crosslinked forms. Exemplary addition polymers tend to be glassy (a Tg greater than room temperature). This is the case for polyvinylchloride and polymethylmethacrylate, polystyrene polymer compositions, or alloys or low in crystallinity for polyvinylidene fluoride and polyvinyl alcohol materials.

In some embodiments of the invention the nanofiber material is a polyamide condensation polymer. In more specific embodiments, the polyamide condensation polymer is a nylon polymer. The term “nylon” is a generic name for all long chain synthetic polyamides. Typically, nylon nomenclature includes a series of numbers such as in nylon-6,6 which indicates that the starting materials are a C₆ diamine and a C₆ diacid (the first digit indicating a C₆ diamine and the second digit indicating a C₆ dicarboxylic acid compound). Another nylon can be made by the polycondensation of epsilon caprolactam in the presence of a small amount of water. This reaction forms a nylon-6 (made from a cyclic lactam—also known as epsilon-aminocaproic acid) that is a linear polyamide. Further, nylon copolymers are also contemplated. Copolymers can be made by combining various diamine compounds, various diacid compounds and various cyclic lactam structures in a reaction mixture and then forming the nylon with randomly positioned monomeric materials in a polyamide structure. For example, a nylon 6,6-6,10 material is a nylon manufactured from hexamethylene diamine and a C₆ and a C₁₀ blend of diacids. A nylon 6-6,6-6,10 is a nylon manufactured by copolymerization of epsilon aminocaproic acid, hexamethylene diamine and a blend of a C₆ and a C₁₀ diacid material.

Block copolymers can also be used as nanofiber materials. In preparing a composition for the preparation of nanofibers, a solvent system can be chosen such that both blocks are soluble in the solvent. One example is an ABA (styrene-EP-styrene) or AB (styrene-EP) polymer in methylene chloride solvent. Examples of such block copolymers are Kraton™ type of AB and ABA block polymers including styrene/butadiene and styrene/hydrogenated butadiene(ethylene propylene), Pebax™ type of epsilon-caprolactam/ethylene oxide, Sympatex™ polyester/ethylene oxide and polyurethanes of ethylene oxide and isocyanates.

Addition polymers such as polyvinylidene fluoride, syndiotactic polystyrene, copolymers of vinylidene fluoride and hexafluoropropylene, polyvinyl alcohol, polyvinyl acetate, amorphous addition polymers, such as poly(acrylonitrile) and its copolymers with acrylic acid and methacrylates, polystyrene, poly(vinyl chloride) and its various copolymers, poly(methyl methacrylate) and its various copolymers, can be solution spun with relative ease because they are soluble at low pressures and temperatures. Highly crystalline polymer like polyethylene and polypropylene generally require higher temperature and high pressure solvents if they are to be solution spun. Electrostatic solution spinning is one method of making nanofibers and microfiber.

Nanofibers can also be formed from polymeric compositions comprising two or more polymeric materials in polymer admixture, alloy format, or in a crosslinked chemically bonded structure. Such polymer compositions can physical properties by changing polymer attributes such as improving polymer chain flexibility or chain mobility, increasing overall molecular weight and providing reinforcement through the formation of networks of polymeric materials.

Two related polymer materials can be blended to provide the nanofiber with beneficial properties. For example, a high molecular weight polyvinylchloride can be blended with a low molecular weight polyvinylchloride. Similarly, a high molecular weight nylon material can be blended with a low molecular weight nylon material. Further, differing species of a general polymeric genus can be blended. For example, a high molecular weight styrene material can be blended with a low molecular weight, high impact polystyrene. A Nylon-6 material can be blended with a nylon copolymer such as a Nylon-6; 6,6; 6,10 copolymer. Further, a polyvinyl alcohol having a low degree of hydrolysis such as a 87% hydrolyzed polyvinyl alcohol can be blended with a fully or super hydrolyzed polyvinyl alcohol having a degree of hydrolysis between 98 and 99.9% and higher. All of these materials in admixture can be crosslinked using appropriate crosslinking mechanisms. Nylons can be crosslinked using crosslinking agents that are reactive with the nitrogen atom in the amide linkage. Polyvinyl alcohol materials can be crosslinked using hydroxyl reactive materials such as monoaldehydes, such as formaldehyde, ureas, melamine-formaldehyde resin and its analogues, boric acids, and other inorganic compounds, dialdehydes, diacids, urethanes, epoxies, and other known crosslinking agents. Crosslinking reagent reacts and forms covalent bonds between polymer chains to substantially improve molecular weight, chemical resistance, overall strength and resistance to mechanical degradation.

Electrospinning produces a population of nanofibers that may differ in diameter, typically from about 5 nm to about 1000 nm.

Nanofibers can be produced by the electrospinning process that uses an electric field to control the formation and deposition of polymers. A polymer solution is injected with an electrical potential. The electrical potential creates a charge imbalance that leads to the ejection of a polymer solution stream from the tip of an emitter such as a needle. The polymer jet within the electric field is directed toward a grounded substrate, during which time the solvent evaporates and fibers are formed. The resulting single continuous filament collects as a nonwoven fabric on a support. Electrospinning processes for the production of nanofibers have been described in U.S. Pat. Nos. 4,650,506 (Barris) and 6,743,273 (Chung et al.).

Electrospun nanofiber networks may be produced having random or directed orientations. Random fibers may be assembled into layered surfaces. In some embodiments, the nanofibers of the invention comprise a random distribution of fine fibers that can be bonded to form an interlocking network. The nanofiber interlocking networks have relatively small spaces between the fibers. Such spaces typically range, between fibers, of about 0.01 to about 25 microns, preferably about 2 to about 10 microns. Such spaces form pores or channels in the nanofiber network allowing for diffusion of ions, metabolites, proteins, and/or bioactive molecules and/or allowing cells to penetrate and permeate the network and grow in an environment that promotes multipoint attachments between cells and the nanofibers.

Nanofiber networks may also be electrospun in an oriented manner. Such oriented electrospinning allows for the fabrication of a nanofiber network comprising a single layer of nanofibers or a single layer formed by a continuous nanofiber wherein the network has a height of the diameter of a single nanofiber. Physical properties including porosity, solidity, fibril density, texture, rugosity, and fiber orientation of the single layer network may be selected by controlling the direction and/or orientation of the nanofiber onto the support during the electrospinning process. Preferably the pore size allows cells to penetrate and/or migrate through the single layer nanofiber network. In an embodiment, the space between fibers is about 0.01 to about 25 microns. In another embodiment, the space between fibers is about 2 to about 10 microns.

The nanofiber may optionally include other materials.

In some aspects, the nanofibers can be formed by combining a polymeric material with an additive composition. The additive composition can influence packing of the polymer such that electrospinning of the polymer results in the production of a population of nanofibers having a greater number or percentage of thin fibers as compared to a population of nanofibers electrospun form a polymer solution not containing the additive. In an embodiment, the polymer solution comprises from about 0.25% to about 15% w/w additive composition. In another embodiment, the polymer solution comprises from about 1% to about 10% w/w additive composition. In some embodiments, the additive composition that influences packing of the polymer includes a bioactive molecule such as a lipid. In some embodiments the lipid can be selected from the group consisting of lysophosphatidylcholine, phosphatidyl-choline, sphingomyelin, cholesterol, and mixtures thereof.

While the polymeric coating on the nanofibers promotes cell attachment, cell attachment may be further improved by engineering the texture and rugosity of the nanofibrillar structure. For example, the nanofibrillar structure may be comprised of multiple nanofibers having different diameters and/or multiple nanofibers fabricated from different polymers. Solidity of the nanofibrillar structure may also be engineered to affect cell growth and/or differentiation. In an embodiment, the nanofibrillar structure has a solidity of about 3 percent to about 70 percent. In another embodiment, the nanofibrillar structure has a solidity of about 3 percent to about 50 percent. In another embodiment, the nanofibrillar structure has a solidity of about 3 percent to about 30 percent. In another embodiment, the nanofibrillar structure has a solidity of about 3 percent to about 10 percent. In another embodiment, the nanofibrillar structure has a solidity of about 3 percent to about 5 percent. In another embodiment, the nanofibrillar structure has a solidity of about 10 percent to about 30 percent.

In some aspects the nanofibers include a fluorescent marker. The fluorescent marker allows, for example, visualization of a nanofiber, identification of specific nanofibers within a nanofiber blend, identification of a chemical or physical property of a nanofiber, and evaluation of the degradation of and/or redistribution of nanofibers and/or structures comprising nanofibers, including multi-layered assemblies useful for engineering tissue. The fluorescent marker may be photobleachable or non-photobleachable. The fluorescent marker may be pH sensitive or pH insensitive. Preferably the fluorescent marker is non-cytotoxic.

The polymers used to form the nanofibers can also have adhering characteristic such that when contacted with a cellulosic, polyvinyl, polyester, polystyrene, or polyamide support, the nanofiber adheres to the support with sufficient strength such that it is securely bonded to the support and can resist delaminating effects associated with mechanical stresses. Adhesion of the nanofiber to the support can arise from solvent effects of fiber formation as the fiber is contacted with the support or the post treatment of the fiber on the support with heat or pressure. Polymers plasticized with solvent or steam at the time of adhesion can have increased adhesion.

The term “nanofibrillar support” as used herein means any surface on which nanofiber or network of nanofibers is deposited. The nanofibrillar support may be any surface that offers structural support for the deposited network of nanofibers. The nanofibrillar support may comprise glass or plastic. Preferably the plastic is non-cytotoxic. In some aspects, the nanofibrillar support may be a film or culture container.

The nanofibrillar support may be water-soluble or water insoluble. A nanofibrillar support that is water-soluble is preferably a polyvinyl alcohol film. In many aspects, and for most methods, the average size of the pores in the nanofibrillar structure is too small to allow for cell entry into the nanofibrillar structure. However, the movement of cells may depend on the size of the cell and the size of the pores in the nanofibrillar structure.

Preferably the pores in a porous nanofibrillar structure have a diameter of about 0.2 μm to about 10 μm. The nanofibrillar structure may be biodegradable and/or biodissolvable. Preferably the nanofibrillar structure is biocompatible.

In some aspects, the nanofibrillar support is selected from glass substrates, such as glass microscope slides, glass cover slips, and glass films; polymer substrates, such as polymer films; and other biomaterial substrates suitable for cell culturing. The nanofibrillar support can have certain properties useful for the useful for the preparation and/or use of a cell culture device. In some specific aspects the nanofibrillar support has one or more properties such as manipulability and/or flexibility, resistance to sterilization, resistance to degradation by radiation, chemical inertness, transparency, non-flammability, and smooth surface properties. In some specific aspects, the nanofibrillar support includes a halogenated thermoplastic resin, such as halogenated fluorinated-chlorinated resins. Specifically, the support can include chlorotrifluoroethylene (CTFE). In other specific aspects, the support is about 0.25 mm or less.

The term “spacer” as used herein means a layer separating a nanofiber or nanofiber network from a surface of a support or a surface of another nanofibrillar structure such that the structures are separated by the diameter or thickness of the spacer. The spacer may comprise a polymer fine fiber or film. Preferably the film has a thickness of about 10 microns to about 50 microns. The spacer may comprise a polymer including cellulose, starch, polyamide, polyester, or polytetrafluoroethylene. The fine fiber may comprise a microfiber. A microfiber is a polymer fine fiber comprising a diameter of about 1 μm to about 30 μm. The microfiber may be unwoven or net.

In other aspects of the invention, the coating having a polymer including at least one pendent amine group and at least one latent reactive group is formed on other types of nano-structured articles. Other exemplary nano-structured cell culture articles include multi-walled carbon nanotubes (MWCN; see Chen et al. (1998) Science 282:95); hydroxyapatite particulate surfaces (Rosa et al. (2003) Dental Mater. 19:768-772). The polymeric coatings can also be formed over the surface of natural materials, such as self-assembling peptide nanofiber scaffolds (Genove et al. (2005) Biomaterials. 26:3341-3351) and collagen/hyaluronic acid polyelectrolyte multilayers (Zheng et al. (2005) Biomaterials, 26:3353-61). In other cases, the three-dimension surface can be created from stabilized layers of nanoparticles.

The nanofibrillar structure can be associated with or formed on articles such as supports, cell culture articles, and medical devices. Such articles can be combined or fabricated with the nanofibrillar structure to form various articles or assemblies, including cell culture apparatuses and medical devices. Articles such as supports, cell culture articles, and medical devices can be made of the same material as the nanofibrillar structure, or can be made from different materials.

Example of materials which can be used to form an article associated with the nanofibrillar structure, such as a support, cell culture article, or medical device, include synthetic polymers, including oligomers, homopolymers, and copolymers resulting from either addition or condensation polymerizations. Examples of suitable addition polymers include, but are not limited to, acrylics such as those polymerized from methyl acrylate, methyl methacrylate, hydroxyethyl methacrylate, hydroxyethyl acrylate, acrylic acid, methacrylic acid, glyceryl acrylate, glyceryl methacrylate, methacrylamide, and acrylamide; vinyls such as ethylene, propylene, vinyl chloride, and styrene.

Exemplary polymeric materials commonly used in cell culture articles include polystyrene and polypropylene.

Examples of condensation polymers include, but are not limited to, nylons such as polycaprolactam, polylauryl lactam, polyhexamethylene adipamide, and polyhexamethylene dodecanediamide, and also polyurethanes, polycarbonates, polyamides, polysulfones, poly(ethylene terephthalate), polylactic acid, polyglycolic acid, polydimethylsiloxanes, and polyetherketone.

Biodegradable polymers can also be used in the preparation of an article associated with the nanofibrillar structure. Examples of classes of synthetic polymers that have been studied as biodegradable materials include polyesters, polyamides, polyurethanes, polyorthoesters, polycaprolactone (PCL), polyiminocarbonates, aliphatic carbonates, polyphosphazenes, polyanhydrides, and copolymers thereof. Specific examples of biodegradable materials that can be used in connection with, for example, implantable medical devices include polylactide, polygylcolide, polydioxanone, poly(lactide-co-glycolide), poly(glycolide-co-polydioxanone), polyanhydrides, poly(glycolide-co-trimethylene carbonate), and poly(glycolide-co-caprolactone). Blends of these polymers with other biodegradable polymers can also be used.

In some aspects, the nanofibrillar structure can be formed on or associated with a surface of an article that is pre-coated with a polymeric material that is different than the polymer having pendent amine groups. For example, articles can be pre-coated with Parylene or an organosilane material to provide a base coat onto which the nanofibrillar structure can be associated or formed.

The nanofibrillar structure can be formed on or associated with articles fabricated from metals, metal alloys, and ceramics, and in many cases these articles can have a pre-coating of Parylene or an organosilane material. The metals and metal alloys include, but are not limited to, titanium, Nitinol, stainless steel, tantalum, and cobalt chromium. A second class of metals includes the noble metals such as gold, silver, copper, and platinum uridium. The ceramics include, but are not limited to, silicon nitride, silicon carbide, zirconia, and alumina, as well as glass, silica, and sapphire. Combinations of ceramics and metals are another class of biomaterials.

In other aspects of the invention, the polymeric coating is formed on the surface of the nanofibers of a nanofibrillar structure that is associated with or formed on at least a portion of an implantable medical article. This refers to implantable medical devices that include a network of nanofibers that are formed on, or that are in some way associated with an implantable medical article. Such devices can be formed by various processes. One process of forming such a device can include electrospinning nanofibers on all or a portion of an implantable medical device. Another process can include forming a nanofibrillar structure and then attaching the nanofibrillar structure to a portion of an implantable medical device.

Exemplary medical articles include vascular implants and grafts, grafts, surgical devices; synthetic prostheses; vascular prosthesis including endoprosthesis, stent-graft, and endovascular-stent combinations; small diameter grafts, abdominal aortic aneurysm grafts; wound dressings and wound management device; hemostatic barriers; mesh and hernia plugs; patches, including uterine bleeding patches, atrial septic defect (ASD) patches, patent foramen ovale (PFO) patches, ventricular septal defect (VSD) patches, and other generic cardiac patches; ASD, PFO, and VSD closures; percutaneous closure devices, mitral valve repair devices; left atrial appendage filters; valve annuloplasty devices, catheters; central venous access catheters, vascular access catheters, abscess drainage catheters, drug infusion catheters, parental feeding catheters, intravenous catheters (e.g., treated with antithrombotic agents), stroke therapy catheters, blood pressure and stent graft catheters; anastomosis devices and anastomotic closures; aneurysm exclusion devices; biosensors including glucose sensors; birth control devices; breast implants; cardiac sensors; infection control devices; membranes; tissue scaffolds; tissue-related materials; shunts including cerebral spinal fluid (CSF) shunts, glaucoma drain shunts; dental devices and dental implants; ear devices such as ear drainage tubes, tympanostomy vent tubes; ophthalmic devices; cuffs and cuff portions of devices including drainage tube cuffs, implanted drug infusion tube cuffs, catheter cuff, sewing cuff; spinal and neurological devices; nerve regeneration conduits; neurological catheters; neuropatches; orthopedic devices such as orthopedic joint implants, bone repair/augmentation devices, cartilage repair devices; urological devices and urethral devices such as urological implants, bladder devices, renal devices and hemodialysis devices, colostomy bag attachment devices; biliary drainage products.

In some aspects, the nanofibers of the nanofibrillar structure have a coating that includes a non-biodegradable polymer one or more and preferably a plurality of pendent amine groups, and one or preferably more than one pendent latent reactive groups. “Non-biodegradable” refers to polymers that are generally not able to be non-enzymatically, hydrolytically or enzymatically degraded. For example, the non-biodegradable polymer is resistant to degradation that may be caused by proteases. However, it is noted that while the coating includes a non-biodegradable amine-presenting polymer, the coating is not limited to non-biodegradable materials, and therefore may also include biodegradable materials, such as natural or synthetic biodegradable polymers.

The coating includes latent reactive groups wherein at least a portion of the groups are activated during the coating process to bond the polymer to the surface of the nanofibers of the nanofibrillar structure. For purposes of describing the formed coating of invention, the polymer (in the formed coating) that is covalently bonded to the surface of the nanofibers may be referred to as having “latent reacted groups,” or, “reacted groups,” referring to one or more of these latent reactive groups on the polymer has been activated and reacted to form a covalent bond between the polymer and the surface of the nanofiber.

By binding to the surface via the latent reacted groups, the immobilized polymer provides positively-charged amine groups to the surface of the nanofibers of the nanofibrillar structure. It is thought that this binding arrangement allows for the formation of a very durable and effective surface for cell attachment. This surface has been shown to be highly effective for processes including maintenance and differentiation of cells on the nanofibrillar structure.

The plurality of pendent amine groups on the non-biodegradable polymer can provide a positive charge to the coating in pH conditions suitable for cell culture. For example, the non-biodegradable polymer will provide a positive charge to the nanofibers of the nanofibrillar structure in conditions ranging from about pH 5.0 to about pH 10.0.

The non-biodegradable polymer can have primary amine, secondary amine, tertiary amine, or combinations of these amine groups pendent from the polymer.

In one aspect of the invention, an exemplary amine-containing group has the following formula: R₁R₂NR₃R₄, wherein R₁ is: 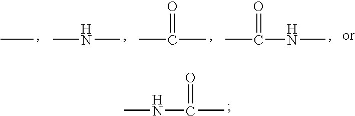
wherein R₂ is C₁-C₈ linear or branched alkyl; and wherein R₃ and R₄ are both attached to the nitrogen and are individually H or C₁-C₆ linear or branched alkyl. As pendent from the polymer, the amine-containing group can be represented by the formula P—[R₁R₂NR₃R₄], P being a portion of the polymeric backbone.

In some more specific aspects, R₁ is 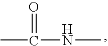
R₂ is C₂-C₄ linear or branched alkyl; and, R₃ and R₄ are both attached to the nitrogen and are individually H, CH₃, or C₂H₅.

Exemplary amine containing groups include those found on polymerizable monomers such as 3-aminopropylmethacrylamide (APMA), 3-aminoethylmethacrylamide (AEMA), dimethylaminopropylmethacrylamide (DMAPMA), and the like. Therefore, in some aspects, polymers including pendent amine groups and latent reactive groups can be formed by copolymerizing a monomer having a group —R₁R₂NR₃R₄ as defined above with a comonomer bearing a latent reactive group. Optionally, other non-amine or non-latent reactive group-containing monomers can be included in the polymer.

In one aspect, the polymer includes an amine-containing group in a molar amount of about 10% or greater, as based on the monomer content of the polymer. This can be achieved, for example, by preparing a polymer with 10% or greater of an amine-containing group monomer. In some aspects, the amine-containing group is present in a molar amount of 50% or greater, 60% or greater, 70% or greater, 80% or greater, 90% or greater, or 95% or greater. In some aspects the polymer includes amine-containing groups in a molar amount in the range of about 90% - 99.95%. An exemplary preparation of a copolymer includes about 98.4% amine-containing monomer, such as APMA, AEMA, or DMAPMA and about 1.6% of monomer including the latent reactive group.

Control over amount of amine group and amount of latent reactive group can be exercised by copolymerizing an amine-containing monomer with a latent reactive group-containing monomer (and optionally a non-amine or non-photo reactive group-containing monomer). Other exemplary amine-containing polymers can be formed by the copolymerization of, for example, amine-containing monomers such as N-(2-amino-2-methylpropyl)methacrylamide, p-aminostyrene, allyl amine, or combinations thereof with a monomer having a pendent latent reactive groups to provide a polymer having pendent amine groups and latent reactive groups. These amine-containing monomers can also be copolymerized with other non-primary amine-containing monomers, such as acrylamide, methacrylamide, vinyl pyrrolidinone, or derivatives thereof, to provide a polymer having desired properties, such as a desired density of amine groups and photoreactive groups. Other suitable polymers that have amine groups include polymers that are formed from monomers such as 2-aminomethylmethacrylate, 3-(aminopropyl)-methacrylamide, and diallylamine. Dendrimers that include photogroups and pendent amine groups can also be used.

In some aspects a polymer having pendent amine groups and hydrophobic properties can be prepared. This can be achieved by one or more schemes for the synthesis of the polymer. For example, a polymer can be formed with a desired amount of hydrophobic monomers, such as (alkyl)acrylate monomers, or the amine-presenting monomers can include longer alkyl chain lengths. For example, any one or more of the groups R₂, R₃, and/or R₄ can include alkyl groups of 3 or more carbon atoms.

Another method for preparing the non-biodegradable polymer includes steps of derivatizing a preformed polymer with a compound that includes a latent reactive group. For example, a homopolymer or heteropolymer having pendent amine groups can be readily derivatized with a photoreactive group by reacting a portion of the pendent amine groups with a compound having a photoreactive group and a group that is reactive with an amine group, such as 4-benzoylbenzoyl chloride.

In some aspects, the polymer having pendent amine groups and at least one latent reactive group is selected from polyethyleneimine, polypropyleneimine, and polyamidoamine. In one specific aspect the polymeric material includes polyethyleneimine with one or more latent reactive group(s).

Latent reactive groups, broadly defined, are groups that respond to specific applied external stimuli to undergo active specie generation with resultant covalent bonding to a target. Latent reactive groups are those groups of atoms in a molecule that retain their covalent bonds unchanged under conditions of storage but which, upon activation, form covalent bonds with other molecules. The latent reactive groups generate active species such as free radicals, nitrenes, carbenes, and excited states of ketones upon absorption of external electromagnetic or kinetic (thermal) energy. Latent reactive groups may be chosen to be responsive to various portions of the electromagnetic spectrum, and latent reactive groups that are responsive to ultraviolet, visible or infrared portions of the spectrum are preferred. Latent reactive groups, including those that are described herein, are well known in the art. See, for example, U.S. Pat. No. 5,002,582 (Guire et al., “Preparation of Polymeric Surfaces Via Covalently Attaching Polymers”). The present invention contemplates the use of any suitable latent reactive group for formation of the inventive coatings as described herein.

Photoreactive groups can generate active species such as free radicals and particularly nitrenes, carbenes, and excited states of ketones, upon absorption of electromagnetic energy. Photoreactive groups can be chosen to be responsive to various portions of the electromagnetic spectrum, and that are responsive to the ultraviolet and visible portions of the spectrum are preferred.

Photoreactive aryl ketones are preferred, such as acetophenone, benzophenone, anthraquinone, anthrone, and anthrone-like heterocycles (for example, heterocyclic analogs of anthrone such as those having nitrogen, oxygen, or sulfur in the 10-position), or their substituted (for example, ring substituted) derivatives. Examples of preferred aryl ketones include heterocyclic derivatives of anthrone, including acridone, xanthone, and thioxanthone, and their ring substituted derivatives. Some preferred photoreactive groups are thioxanthone, and its derivatives, having excitation energies greater than about 360 nm.

The functional groups of such ketones are preferred since they are readily capable of undergoing the activation/inactivation/reactivation cycle described herein. Benzophenone is a particularly preferred latent reactive moiety, since it is capable of photochemical excitation with the initial formation of an excited singlet state that undergoes intersystem crossing to the triplet state. The excited triplet state can insert into carbon-hydrogen bonds by abstraction of a hydrogen atom (from a support surface, for example), thus creating a radical pair. Subsequent collapse of the radical pair leads to formation of a new carbon-carbon bond. If a reactive bond (for example, carbon-hydrogen) is not available for bonding, the ultraviolet light-induced excitation of the benzophenone group is reversible and the molecule returns to ground state energy level upon removal of the energy source. Photoactivatable aryl ketones such as benzophenone and acetophenone are of particular importance inasmuch as these groups are subject to multiple reactivation in water and hence provide increased coating efficiency.

The azides constitute another class of photoreactive groups and include arylazides (C₆R₅N₃) such as phenyl azide and 4-fluoro-3-nitrophenyl azide; acyl azides (—CO—N₃) such as benzoyl azide and p-methylbenzoyl azide; azido formates (—O—CON₃) such as ethyl azidoformate and phenyl azidoformate; sulfonyl azides (—SO₂—N₃) such as benezensulfonyl azide; and phosphoryl azides [(RO)₂PON₃] such as diphenyl phosphoryl azide and diethyl phosphoryl azide.

Diazo compounds constitute another class of photoreactive groups and include diazoalkanes (—CHN₂) such as diazomethane and diphenyldiazomethane; diazoketones (—CO—CHN₂) such as diazoacetophenone and 1-trifluoromethyl-1-diazo-2-pentanone; diazoacetates (—O—COCHN₂) such as t-butyl diazoacetate and phenyl diazoacetate; and beta-keto-alpha-diazoacetatoacetates (—CO—CN₂COO—) such as t-butyl alpha diazoacetoacetate.

Other photoreactive groups include the diazirines (—CHN₂) such as 3-trifluoromethyl-3-phenyldiazirine; and ketenes (CH═C═O) such as ketene and diphenylketene.

Peroxy compounds are contemplated as another class of latent reactive groups and include dialkyl peroxides such as di-t-butyl peroxide and dicyclohexyl peroxide and diacyl peroxides such as dibenzoyl peroxide and diacetyl peroxide and peroxyesters such as ethyl peroxybenzoate.

In some aspects, the latent reactive group is present in a molar amount (relative to the monomers of the polymer) of up to about 10%, or an amount of up to about 5%. In some aspects, the polymer includes the latent reactive group in a molar amount the range of about 0.05%-10%. An exemplary preparation of a copolymer includes about 98.4% amine-containing monomer, such as APMA, AEMA, or DMAPMA and about 1.6% of monomer including the latent reactive group.

The inventive coating formed on the surface of the nanofibers of the nanofibrillar structure using any suitable method. As described above, a polymer having pendent amine groups and pendent latent reactive groups can be disposed on the surface of the nanofibers and the surface can be treated to activate the latent reactive groups thereby bonding the polymer to the surface of the nanofibers, and forming a thin polymeric coating over the nanofiber surface.

In another method, the polymer is formed on the nanofiber surface of the nanofibrillar structure by a graft polymerization method. For example, a monomer including a latent reactive group and a polymerizable group can be disposed and bonded to the surface of the nanofibers. A composition of monomers including amine groups can then be disposed on the surface, and a polymerization reaction can be initiated to cause the formation of a polymer chain from and bonded to the surface of the nanofibrillar structure.

In yet another method, the coating can be formed using a crosslinking agent having two or more latent reactive groups, wherein the crosslinking agent is used to bond the polymer to the surface of the nanofibers of the nanofibrillar structure. The crosslinking agent can have any two or more of the latent reactive groups as described herein. In forming the polymeric coating, the crosslinking agent can be disposed on the surface of the nanofibrillar structure followed by disposing the polymer having pendent amine groups, or the crosslinking agent can be disposed in combination with the polymer, or both.

If photoreactive groups are present on the cross-linking agent, preferably they are adapted to undergo reversible photolytic homolysis, thereby permitting photoreactive groups that are not consumed in attachment to a polymeric material to revert to an inactive, or “latent” state. These photoreactive groups can be subsequently activated, in order to attach to the polymer with an abstractable hydrogen for covalent bond formation. Thus, excitation of the photo reactive group is reversible and the group can return to a ground state energy level upon removal of the energy source. In some embodiments, preferred cross-linking agents are those groups that can be subject to multiple activations and hence provide increased coating efficiency. Exemplary crosslinking agents are described in Applicant's U.S. Pat. No. 5,414,075 (Swan et al.), and U.S. Publication No. 2003/0165613 Al (Chappa et al.). See also U.S. Pat. Nos. 5,714,360 (Swan et al.) and 5,637,460 (Swan et al.).

The non-biodegradable polymer having pendent amine groups can be bonded to the nanofibers of a nanofibrillar structure either alone or with other optional components. In its simplest form, the coating composition consists of, for example, (i) a non-biodegradable polymer having at least one, or preferably a plurality of pendent amine groups, and least one latent reactive group and/or (ii) a non-biodegradable polymer having at least one, or preferably a plurality of pendent amine groups and a crosslinking agent having two or more or more latent reactive groups. Other components may be added to the coating composition to change or improve aspects of the coating. The components may be polymeric or non-polymeric components.

Other synthetic or natural, biodegradable or non-biodegradable polymers can be added to the composition to form the coating. A “synthetic polymer” refers to a polymer that is synthetically prepared and that includes non-naturally occurring monomeric units. For example, a synthetic polymer can include non-natural monomeric units such as acrylate, acrylamide, etc. Synthetic polymers are typically formed by traditional polymerization reactions, such as addition, condensation, or free-radical polymerizations. Synthetic polymers can also include those having natural monomeric units, such as naturally-occurring peptide, nucleotide, and saccharide monomeric units in combination with non-natural monomeric units (for example synthetic peptide, nucleotide, and saccharide derivatives). These types of synthetic polymers can be produced by standard synthetic techniques, such as by solid phase synthesis, or recombinantly, when allowed.

A “natural polymer” refers to a polymer that is either naturally, recombinantly, or synthetically prepared and that consists of naturally occurring monomeric units in the polymeric backbone. In some cases, the natural polymer may be modified, processed, dervitized, or otherwise treated to change the chemical and/or physical properties of the natural polymer. In these instances, the term “natural polymer” will be modified to reflect the change to the natural polymer (for example, a “derivitized natural polymer”, or a “deglycosylated natural polymer”).

Biodegradable materials, such as biodegradable polymers, can also be present in the coating. The biodegradable materials can optionally be present in the same coated layer as the non-biodegradable amine-presenting polymer, can be present in another coated layer, if included in the coating, or both. For example, a coated layer that includes a biodegradable polymer can be formed between the coated layer that includes the non-biodegradable amine-presenting polymer and the article surface, or can be formed on top of the coated layer that includes the non-biodegradable amine-presenting polymer. During culturing, the biodegradable polymer can degrade while the non-biodegradable polymer remains present in the coating and provides an adherent surface during protracted periods of culturing.

In some aspects of the invention, a “bioactive molecule” can be associated with the polymer-coated nanofibrillar structure. For example, one or more bioactive molecules can be present in the nanofiber, and/or in the coating on the nanofibers that contains the non-biodegradable polymer having pendent amine groups and latent reactive groups. In some cases the bioactive molecule can be a biodegradable material, as described herein. While one or more bioactive molecule(s) can be associated with the polymer-coated nanofibrillar structure, bioactive molecule(s) may also be included in liquid media when cell culture methods are performed in conjunction with the polymer-coated nanofibrillar structure. Therefore, recitation of bioactive molecules is not intended to limit the presence of the molecule to the coating or to any media, unless specifically described herein.

The term “bioactive molecule” as used herein means a molecule that has an effect on a cell or tissue. The term includes human or veterinary therapeutics, nutraceuticals, vitamins, salts, electrolytes, amino acids, peptides, polypeptides, proteins, carbohydrates, lipids, polysaccharides, nucleic acids, nucleotides, polynucelotides, glycoproteins, lipoproteins, glycolipids, glycosaminoglycans, proteoglycans, growth factors, differentiation factors, hormones, neurotransmitters, pheromones, chalones, prostaglandins, immunoglobulins, monokines and other cytokines, humectants, minerals, electrically and magnetically reactive materials, light sensitive materials, anti-oxidants, molecules that may be metabolized as a source of cellular energy, antigens, and any molecules that can cause a cellular or physiological response. Any combination of molecules can be used, as well as agonists or antagonists of these molecules. Glycoaminoglycans include glycoproteins, proteoglycans, and hyaluronan. Polysaccharides include cellulose, starch, alginic acid, chitosan, or hyaluronan. Cytokines include, but are not limited to, cardiotrophin, stromal cell derived factor, macrophage derived chemokine (MDC), melanoma growth stimulatory activity (MGSA), macrophage inflammatory proteins 1 alpha (MIP-1 alpha), 2, 3 alpha, 3 beta, 4 and 5, interleukin (IL) 1, IL-2, IL-3, IL-4, IL-5, IL-6, IL-7, IL-8, IL-9, IL-10, IL-I 1, IL-12, IL-13, TNF-alpha, and TNF-beta. Immunoglobulins useful in the present invention include, but are not limited to, IgG, IgA, IgM, IgD, IgE, and mixtures thereof. Amino acids, peptides, polypeptides, and proteins may include any type of such molecules of any size and complexity as well as combinations of such molecules. Examples include, but are not limited to, structural proteins, enzymes, and peptide hormones.

The term bioactive molecule also includes fibrous proteins, adhesion proteins, adhesive compounds, deadhesive compounds and targeting compounds. Fibrous proteins include collagen and elastin. Adhesion/deadhesion compounds include fibronectin, laminin, thrombospondin and tenascin C. Adhesive proteins include actin, fibrin, fibrinogen, fibronectin, vitronectin, laminin, cadherins, selectins, intracellular adhesion molecules 1, 2, and 3, and cell-matrix adhesion receptors including but not limited to integrins such as α₅β₁, α₆β₁, α₇β₁, α₄β₂, α₂β₃, α₆β₄.

In some aspects, polymers that have traditionally been used to form coatings for cell attachment can be included in the coating composition. For example, polypeptide-based polymers such as polylysine, collagen, fibronectin, integrin, and laminin can be included in the coatings. Peptide portions of these polypeptides can also be included in the coating composition. Exemplary binding domain sequences of matrix proteins are shown in Table 1.

TABLE 1

| | | |
|---|---|---|
| Fibronectin: | RGDS LDV | REDV |
| Vitronectin | RGDV | |
| Laminin A | LRGDN IKVAV | |
| Laminin B | YIGSR PDSGR | |
| Laminin B2 | RNIAEIIKDA | |
| Collagen I | RGDT DGEA | GTPGPQGIAGQRGVV |
| Thrombospondin | RGD VTXG | FYVVMWK |

Depending on the reagents present in the coating composition, these polypeptide-based polymers can be in an underivitized or derivitized form. For example, the polypeptide-based polymers can be derivitized with latent reactive groups, and then can be activated along with the latent reactive groups pendent from the non-biodegradable polymer to form the coating. Exemplary combinations can include photo-poly(aminopropylmethacrylamide) or photo-poly(ethyleneimine) with one or more of photo-polylysine, photo-collagen, photo-fibronectin, and photo-laminin, or photo-derivitized portions of polypeptides, including those described herein.

Photoderivatized polypeptides, such as collagen, fibronectin, and laminin can be prepared as described in U.S. Pat. No. 5,744,515 (Clapper, Method and Implantable Article for Promoting Endothelialization). As described in this patent, a heterobi-functional agent can be used to photoderivatize a protein. The agent includes a benzophenone photoactivatable group on one end (benzoyl benzoic acid, BBA), a spacer in the middle (epsilon aminocaproic acid, EAC), and an amine reactive thermochemical coupling group on the other end (N-oxysuccinimide, NOS). BBA-EAC is synthesized from 4-benzoylbenzoyl chloride and 6-aminocaproic acid. Then the NOS ester of BBA-EAC is synthesized by esterifying the carboxy group of BBA-EAC by carbodiimide activation with N-hydroxysuccimide to yield BBA-EAC-NOS. Proteins, such as collagen, fibronectin, laminin, and the like can be obtained from commercial sources. The protein is photoderivatized by adding the BBA-EAC-NOS crosslinking agent at a ratio of 10-15 moles of BBA-EAC-NOS per mole of protein.

Bioactive molecules also include leptin, leukemia inhibitory factor (LIF), RGD peptide, tumor necrosis factor alpha and beta, endostatin, angiostatin, thrombospondin, osteogenic protein-1, bone morphogenic proteins 2 and 7, osteonectin, somatomedin-like peptide, osteocalcin, interferon alpha, interferon alpha A, interferon beta, interferon gamma, interferon 1 alpha, and interleukins 2, 3, 4, 5 6, 7, 8, 9, 10, 11, 12, 13, 15, 16, 17 and 18.

The term “growth factor” as used herein means a bioactive molecule that promotes the proliferation of a cell or tissue. Growth factors useful in the present invention include, but are not limited to, transforming growth factor-alpha. (TGF-α), transforming growth factor-beta. (TGF-β), platelet-derived growth factors including the AA, AB and BB isoforms (PDGF), fibroblast growth factors (FGF), including FGF acidic isoforms 1 and 2, FGF basic form 2, and FGF 4, 8, 9 and 10, nerve growth factors (NGF) including NGF 2.5s, NGF 7.0s and beta NGF and neurotrophins, brain derived neurotrophic factor, cartilage derived factor, bone growth factors (BGF), basic fibroblast growth factor, insulin-like growth factor (IGF), vascular endothelial growth factor (VEGF), EG-VEGF, VEGF-related protein, Bv8, VEGF-E, granulocyte colony stimulating factor (G-CSF), insulin like growth factor (IGF) I and II, hepatocyte growth factor, glial neurotrophic growth factor (GDNF), stem cell factor (SCF), keratinocyte growth factor (KGF), transforming growth factors (TGF), including TGFs alpha, beta, betal, beta2, and beta3, skeletal growth factor, bone matrix derived growth factors, and bone derived growth factors and mixtures thereof. Some growth factors may also promote differentiation of a cell or tissue. TGF, for example, may promote growth and/or differentiation of a cell or tissue. Some preferred growth factors include VEGF, NGFs, PDGF-AA, PDGF-BB, PDGF-AB, FGFb, FGFa, and BGF.

The term “differentiation factor” as used herein means a bioactive molecule that promotes the differentiation of cells. The term includes, but is not limited to, neurotrophin, colony stimulating factor (CSF), or transforming growth factor. CSF includes granulocyte-CSF, macrophage-CSF, granulocyte-macrophage-CSF, erythropoietin, and IL-3. Some differentiation factors may also promote the growth of a cell or tissue. TGF and IL-3, for example, may promote differentiation and/or growth of cells.

In some aspects, if other optional components are added to the coating composition, it is generally desirable that the non-biodegradable polymer is the primary component in the composition. If the coating includes some biodegradable components, these components may degrade over a period of time, yet leaving the non-biodegradable polymer as the primary component of the coating.

The reagents of the coating composition, such as the polymeric materials, can be prepared in a suitable liquid, such as an aqueous or alcohol-based liquid. For example, the polymeric materials can be dissolved at concentrations in the range of about 0.1 mg/mL to about 50 mg/mL. However, more typically used concentrations are in the range of about 1 mg/mL to about 10 mg/mL.

The coating can be formed by any suitable method including dip coating, in-solution coating, and spray coating.

In the case wherein the coating included photoreactive groups, generally, the step of irradiating can be performed by subjecting the photoreactive groups to actinic radiation in an amount that promotes activation of the photoreactive group and bonding to the nanofibers of a nanofibrillar structure.

Actinic radiation can be provided by any suitable light source that promotes activation of the photoreactive groups. Preferred light sources (such as those available from Dymax Corp.) provide UV irradiation in the range of 190 nm to 360 nm. A suitable dose of radiation is in the range from about 0.1 mW/cm² to about 20 mW/cm² as measured using a radiometer fitted with a 335 nm band pass filter with a bandwidth of approximately 10 nm.

In some aspects, it may be desirable to use filters in connection with the step of activating the photoreactive groups. The use of filters can be beneficial from the standpoint that they can selectively minimize the amount of radiation of a particular wavelength or wavelengths that are provided to the coating during the activation process. This can be beneficial if one or more components of the coating are sensitive to radiation of a particular wavelength(s), and that may degrade or decompose upon exposure.

Typically, filters are identified by the wavelength of light that is permitted to pass through the filter. Two illustrative types of filters that can be used in connection with the invention are cut-off filters and band pass filters. Generally, cut-off filters are categorized by a cut-off transmittance, at which the light transmittance is approximately 25% of the maximum transmittance. For band pass filters, a range of wavelength is identified for the filter, and the center wavelength is the midpoint of the wavelengths allowed through the filter.

Following the preparation of the coated nanofibrillar structure, a washing step can be performed to remove any excess materials that may not be covalently bonded to the surface of the nanofibers. The coated nanofibrillar structure can also be treated to sterilize the nanofibrillar structure, by, for example, further UV irradiation.

Some advantages of the present invention are related to forming a coating that is particularly useful for providing an adherent coating for a wide variety of cell types. Because of the stability of the coating, the coating process can be performed to provide a coated nanofibrillar structure, and then the coated nanofibrillar structure can be delivered to a user or stored for a period of time before use. In other cases, the coating reagents (including at least the non-biodegradable polymer) can be supplied in a kit to a user, who then can perform the coating process on a nanofibrillar structure. Therefore, the invention also provides kits for preparing coatings including a non-biodegradable polymer. The kits can include instructions for forming the coating, and optionally can include methods for culturing cells using a nanofibrillar structure-that is coated with the reagents of the kit.

In some aspects, the present invention provides coatings and methods for culturing cells using coated nanofibrillar structures, wherein the coated nanofibrillar structures provide an excellent substrates for cell attachment and that can be used in methods wherein the cells can be kept in contact with the coated nanofibrillar structure for a considerable period of time, such as greater than 14 days, greater than 21 days, greater than 28 days, greater than 35 days, greater than 42 days, greater than 49 days, or greater than 56 days. In some aspects, the cells also may be kept in culture for a time period in the range of about 14 to about 60 days. For example, the cells may be disposed on a coated nanofibrillar structure, wherein the cells adhere to the points of the coated structure and are kept viable in the presence of appropriate media. In some cases the cells may expand by proliferation, but, generally, the phenotype of the cells does not change.

In conjunction with the inventive coating, the cells are typically cultured in a liquid media that is suitable for maintaining cells or promoting the formation of a desired cell type. Various base liquid medias may be used, such as RPMI, which can be supplemented with serum, amino acids, trace elements, hormones, antibiotics, salts, buffers, growth factors (such as those described herein), and/or differentiation factors (such as those described herein).

Factors that can affect aspects of cellular function, including growth and differentiation can also be added to the liquid media. These factors can include neurotrophins, cytokines (such as interleukins), insulin-like growth factors, transforming growth factors, epidermal growth factors, fibroblast growth factors, heparin-binding growth factors, tyrosine kinase receptor ligands, platelet derived and vascular endothelial growth factors, and semaphorins.

Exemplary neurotrophins include nerve growth factor (NGF), neurotrophin, and brain-derived neurotrophic factor; exemplary epidermal growth factors include neuregulin, transforming growth factor α, and netrin; exemplary cytokines include interleukin (IL)-2, IL-3, IL-4, IL-5, IL-6, IL-7, IL-9, IL-11, IL-12, IL-13, IL-15, and G-CSF, leukemia inhibitory factor, ciliary neurotrophic factor (CNTF), cardiotrophin-1, and oncostatin-M; exemplary transforming growth factors include glial-derived neurotrophic factor (GDNF), artemin, neurturin, and persephin.

In some aspects of the invention, the method includes culturing stem cells on the coated as described herein in the presence of appropriate media. Stem cells are multi-potent and plastic, which enables them to be induced to differentiate into various cell types. Stem cells include embryonic stem cells, such those obtained from blastocysts, and adult stem cells, which can be obtained from various tissues in an adult body, such as the bone marrow, which provides a source of hematopoietic stem cells. Embryonic stem cells have essentially unlimited proliferation capacity in vitro and therefore can be expanded greatly for applications, such as those involving tissue regeneration. The coated nanofibrillar structures of the present invention provide ideal substrates for culturing these cells, as the cells can be maintained and expanded on coated nanofibrillar structures for considerably longer than on other substrates. Therefore, the coated nanofibrillar structure can greatly facilitate obtaining a great number of stem cells for a desired application such as cell-transplantation or tissue engineering.

Cells cultured according to the processes of the present invention can also be used for drug discovery, gene identification, and for antibody production.

The coated nanofibrillar structures of the present invention also allow clonal or a small number of cells to be seeded in the coated article vessel, and also allow a longer period before the cells have to be harvested, split, or diluted before a confluent state in culture is reached.

In some cases, prior to disposing on the coated nanofibrillar surface, the cells may be kept on a feeder layer of cells. Following culturing for a period of time on the feeder layer, the cells may be transferred to a nanofibrillar cell culture article having the inventive coatings as described herein. According to the invention, it has been discovered that the cells can be cultured for a period of up to about 30 days on the coated nanofibrillar structures without the need to provide a fresh-coated surface.

In other aspects of the invention, the invention relates to a method for the differentiation of neural precursors and stem cells. According to the invention, neural precursors can be cultured in the presence of the inventive coatings and one or more factors, such as neurotrophic growth factors, which induce a morphological or biochemical change characteristic of a partial or fully matured neuronal phenotype.

More specifically, in some aspects, the coated nanofibrillar structures can be used to culture multipotent neuroepithelial stem cells and lineage-restricted intermediate precursor cells which can be induced to differentiate into oligodendrocytes, astrocytes, and neurons. Such precursor cells are present in the CNS at various developmental stages.

In one aspect of the invention, the method is used to promote neurite extension.

PC12 cells (Pheochromocytoma cells), which weakly adhere to plastic, were able to demonstrate excellent adhesion to the coated nanofibrillar structure described herein. Generally PC12 cells are slow growing and can be differentiated with NGF and cAMP acting synergistically. Once differentiated PC12 cells can be maintained for about 14 days. Dexamethasone induces differentiation of a non-neural lineage. Results described herein also show that neural precursor PC12 cells in the presence of the nanofibrillar structure with the non-biodegradable polymeric amine coatings and Nerve Growth Factor (NGF) exhibit neurite extension and the expression of biochemical markers of the sympathetic neuronal phenotypes.

### EXAMPLE 1

### Photo-Polymeric Reagents

1. Photo-poly(APMA) The preparation of photo-poly(aminopropylmeth-acrylamide) (photo-poly(APMA)/APO2) was carried out by the copolymerization of N-(3-aminopropyl)methacrylamide hydrochloride (APMA-HCl) and N-[3-(4-Benzoylbenzamido)propyl]methacrylamide (BBA-APMA), the preparation of which are described in Examples 2 and 3, respectively, of U.S. Pat. No. 5,858,653.

Copolymerization was carried out by adding to a 2 L flask 2.378 g of BBA-APMA (6.7877 mmol), 0.849 of 2,2′-azobis(2-methyl-propionitrile)(AIBN)(5.1748 mmol), and 0.849 g of N,N,N′,N′-tetramethylethylenediamine (TEMED) (6.77 mmol), and then 786 g of dimethylsulfoxide (DMSO) to dissolve the ingredients. The contents were then stirred and deoxygenated with a helium sparge for at least 5 minutes. In a separate flask was dissolved 72.4 g of APMA-HCl (405.215 mmol) in 306 g of DI water with nitrogen sparge. The dissolved APMA-HCl was transferred to the mixture containing BBA-APMA followed by helium sparge for at least 10 minutes. The sealed vessel was then heated overnight at 55° C. to complete the polymerization.

The polymer solution in an amount of 180 mL was then diluted with 180 mL of DI water and dialyzed against deionized water using 12,000-14,000 molecular weight cutoff tubing for at least 96 hours in a 55 gallon tank using a constant flow of 1.25 to 0.35 gallons per minute.

Various coating solutions were prepared with the photo-poly(APMA) polymer ranging from 10 μg/mL to 20 mg/mL in water.

II-IV. Photo-collagen, photo-fibronectin, andphoto-laminin Photo-collagen, photo-fibronectin, and photo-laminin were prepared as described in Example 1 of U.S. Pat. No. 5,744,515.

Various photo-laminin coating solutions were prepared ranging from 25 μg/mL to 300 μg/mL in water.

A photo-fibronectin coating solution was prepared at 25 μg/mL in water.

A photo-collagen coating solution was prepared at 25 μg/mL in 0.012 N HCl.

V. Photo-PEI Photo-PEI was prepared by first drying polyethylenimine (PEI; 24.2 wt. % solids; 2000 kg/mol Mw; BASF Corp.) under vacuum, and then dissolving 1.09 g of PEI in a 19 mL of 90:10 (v/v) chloroform:methanol solution. The PEI solution was then chilled to 0° C, in an ice bath. In 2.8 mL chloroform was added 62 mg BBA-Cl (4-benzoylbenzoyl chloride; the preparation of which is described in U.S. Pat. No. 5,858,653) which was allowed to dissolve. The BBA-Cl solution was added to the chilled, stirring PEI solution. The reaction solution was stirred overnight while warming to room temperature (TLC analysis of the reaction solution revealed no unreacted BBA-Cl present after 2.5 hrs.). The next day the reaction solution was transferred into a large flask and one equivalent of concentrated hydrochloric acid was added along with 77.5 mL deionized water. The organic solvents were removed under vacuum at 40° C. until the aqueous PEI solution was clear in appearance. The aqueous PEI solution was then diluted to a final concentration of 5 mg/mL for use as a coating solution.

V. Photo-RGD Photo-RGD was prepared as described in Example 1 of U.S. Pat. No. 6,121,027. A photo-RGD coating solution was prepared at 25 μg/mL in water.

### EXAMPLE 2

### Substrate Coating

The photo-polymeric reagents prepared in Example 1 of above were coated onto flat (multi-well plates) and three-dimensional substrates (polymeric nanofibers).

In order to coat flat surfaces, coating solutions as described in Example I in an amount of 1.0 mL were added to wells of 12 well plates (polystyrene; Coming). For substrate coating the depth of the coating solution (the distance from the surface of the solution to the surface of the substrate, either polystyrene or nanofiber) is generally 5 mm or less, and typically in the range of about 1 or 2 mm. A Dymax™ lamp was used to deliver 200-300 mJ of energy as measured using a 335 nm band pass filter with a 10 nm bandwidth (on average, the wells were irradiated for about 3-4 minutes with the lamp held at a distance of 20 cm from the wells). The wells were then washed with buffered saline pH 7.2 to remove any unbound reagents. The wells were then UV illuminated again to sterilize the wells using the same illumination conditions as described above.

Uncoated 12 well plates were used as controls.

In order to coat three-dimensional surfaces, coating solutions as described in Example 1 in an amount of 0.5 mL were added to disc nanofiber substrates (Synthetic-ECM™, Donaldson Co., MN; see, e.g., U.S. Patent Pub. No. 2005/0095695). The substrates were irradiated with the Dymax™ lamp at a distance of about 20 cm for 1 minute. The discs were then washed four times with water. The nanofiber substrates were then UV illuminated again to sterilize the nanofiber substrates.

Uncoated nanofiber substrates were used as controls.

### EXAMPLE 3

### Attachment Assay of PC12 Cells on Photo-Polymer Coated Substrates

An attachment assay was performed to determine the effects of plating poorly adherent cells (PC12 cells) on various photopolymer substrates. Rat PC12 (pheochromocytoma) cells obtained from ATCC (accession # CRL 1721) were pre-cultured in collagen-coated polystyrene flasks (15 μg/mL, Sigma) in RPMI medium (Invitrogen) containing 10% horse serum, 5% fetal bovine serum, 2 mM Glutamax (Invitrogen), 1 mM sodium pyruvate (Invitrogen), and 10 mM HEPES (Invitrogen). Cells were incubated at 37° C. in 5% CO₂/95% air humidified chamber. The media was changed every second day. Cells were trypsinized and passaged when they reached 80% confluency. These culture conditions were followed prior to plating the cells into the 12 well substrates having been coated according to the processes as described in Example 2.

PC12 cells between passage #2 and passage #10 were used for all experiments performed. The cells were trypsinized and seeded at a density of 500,000 cells/well in a 12 well plate in RPMI media at a concentration of 500,000 cells/mL. Cells were incubated in a humidified chamber at 37° C. with 5% CO₂ for 48 hours. The cells were at least 99% viable with polygonal morphology prior to plating.

For seeding cells onto nanofibers, the PC12 cells were trypsinized and resuspended in 200 μL of RPMI media. The cell suspension at a density of 500,000 cells/18 mm was carefully added to the coated and uncoated nanofibers (Synthetic-ECM™, product number P609192, Donaldson Co., MN) and the cells were allowed to adhere to the nanofibers for 10 minutes at room temperature under the laminar flow hood. After 10 minutes 800 μL of growth media was gently added around the nanofibers and the cells were placed in a humidified, 5% CO₂/95% air chamber at 37° C. The media was changed every second day.

MTT Attachment Assay After 48 hours, the growth media was removed and the cells growing on coated and uncoated polystyrene, coated and uncoated nanofibers were washed 4 times with Ca⁺⁺- and Mg⁺⁺-free PBS. These multiple washes removed the loosely bound and unbound cells from the wells. On coated substrates 5% cells were removed by the washes whereas on uncoated substrates 70% were removed by the washes. Cells were then incubated for two hours with MTT in humidified chamber at 37° C. (diluted 1:1 with growth media, Sigma). Media containing MTT was removed and the cells were washed again with PBS to get rid of phenol red. 500 μl of dye solubilizer (a mixture of 0.5 ml of 0.04N HCl/isopropyl alcohol and 0.12 ml of 3% SDS/water) was added and the wells were gently rocked at 30 rpm for 30 minutes (or until the dye completely solubilized) at room temperature. The samples were transferred to a 96 well dish and the absorbance was read at 570 nm in a Spectrophotometer (Spectramax, Molecular Devices).

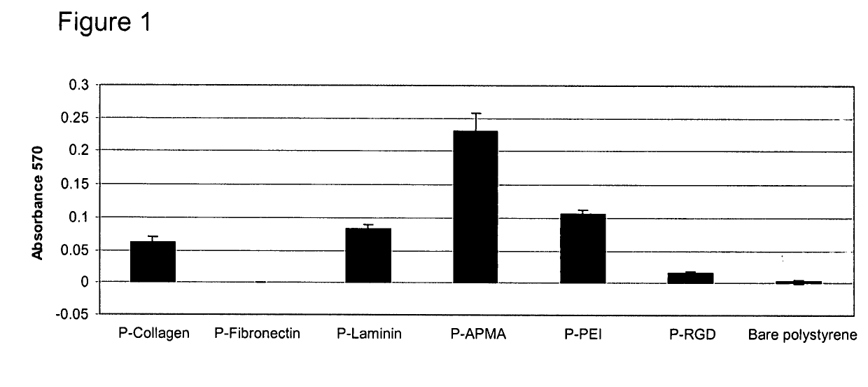

Results of cell attachment on coated and uncoated flat surfaces (12 well plates) are shown in FIG. 1. The best attachment of the PC12 cells was demonstrated in wells that were coated with photo-poly(APMA) followed by photo-laminin and photo-PEI reagents. Uncoated wells and photo-fibronectin coated substrates were used as controls. PC12 cells express a low level of fibronectin receptors on their surface relative to other cell types that adhere well to fibronectin. (see Tomaselli and Reichardt (1987) J. Cell Biol., 105:2347-2358). The results show that photo-RGD and photo-collagen did not provide coatings that performed as well as photo-poly(AMPA), with approximately a two fold difference in the attachment capacity of photo-poly(AMPA) compared to photo-collagen and photo-laminin.

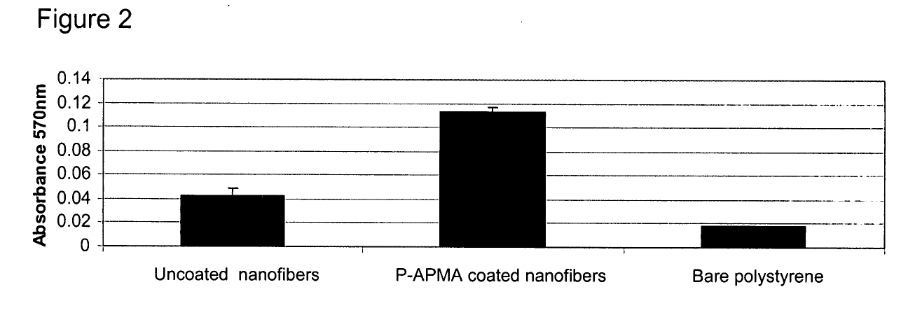

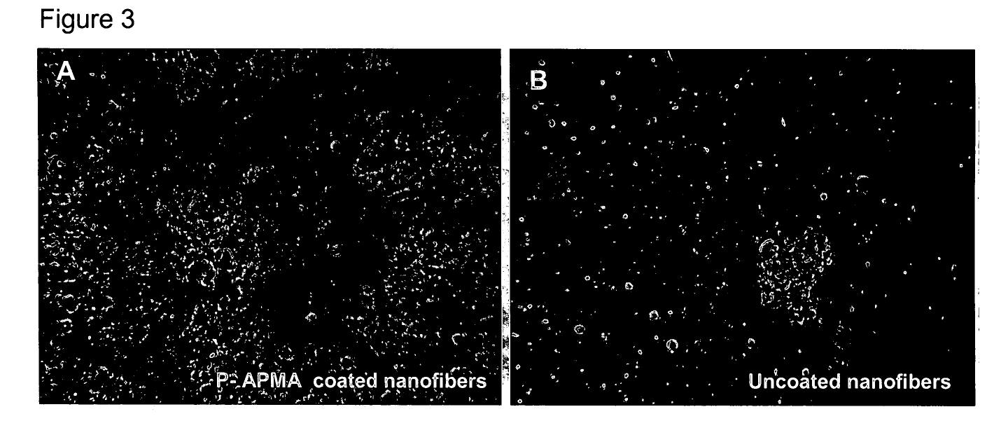

Results of cell attachment on three dimensional surfaces (nanofibers) are shown in FIG. 2. Similar to results on flat surfaces, the photo-poly(APMA)-coated nanofibers showed firm attachment of weakly anchoring cells as compared to uncoated substrates. FIG. 3 shows a bright field microscopic image of PC12 cells growing on photo-poly(APMA)-coated nanofibers (3B) and uncoated nanofibers (3A). After 24 hours the picture image was taken. PC12 cells demonstrated good spreading on the photo-poly(APMA)-coated nanofibers, but on the uncoated surfaces the cells attached to each other to form clusters, but were very weakly attached to the uncoated substrate.

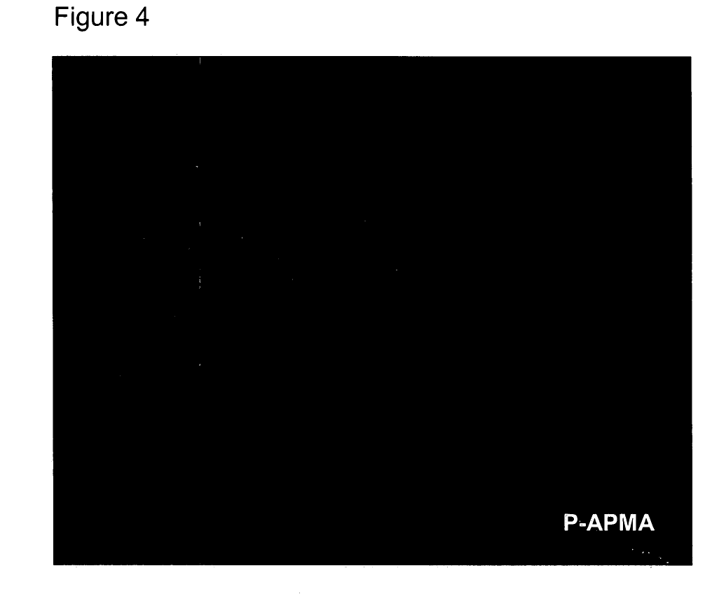

FIG. 4 shows a fluorescence microscopic image of PC 12 cells having been grown on photo-poly(APMA)-coated nanofibers for a period of 24 hours and subject to staining with phalloidin (1:500, Molecular Probes). Phalloidin binds to filamentous actin (F-actin) and provides visualization of the cytoskeletal organization of the cells. The staining results show that the PC 12 cells cultured on photo-poly(APMA)-coated nanofibers retain their normal polygonal morphology showing the strong binding capacity of the substrate does not affect cell morphology. For phalloidin staining, PC12 cells were fixed with 4% paraformaldehyde for 20 min, washed with 0.1 M PBS three times and incubated for an hour at room temperature with phalloidin conjugated with TRITC diluted in PBS containing 0.5% Triton-X-100™ and 5% goat serum. Cells were washed with 0.1 M PBS and observed under an inverted microscope.

The image shows that actin is organized in a cortical ring instead of being highly spread out, when the cells are cultured on three-dimensional substrates. This organization of actin is observed in either tissues or tissue like matrices (Walpita and Hay (2002) Nature Rev. Mol. Cell. Biol., 3:137-141; U.S. Patent Pub. No. 2005/0095695A1).

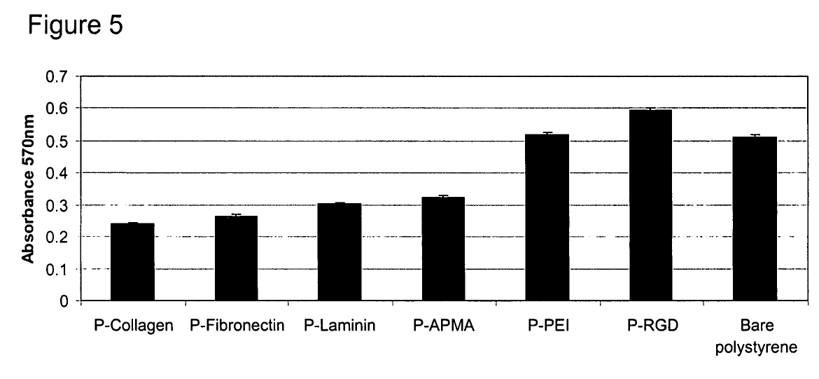

To demonstrate the photo-reagents specifically improved the adhesion of a poorly adherent cell line, a strongly adherent cell line was plated on the photo-polymer coated substrates. Human foreskin fibroblasts (HFF) were plated on photo-polymer coated substrates according to the methods used for the PC12 cells and an MTT attachment assay was performed following culturing. FIG. 5 shows that the presence of the photo-reagents generally did not improve the adherence of the HFF cells in comparison to bare polystyrene. No benefits were observed in the attachment of a strongly adherent cell line on various coated substrates tested.

### EXAMPLE 4

### Proliferation of PC12 Cells on Photo-Polymer Coated Surfaces

PC12 cells were cultured as described in Example 3. BrdU incorporation was tested for cells grown on coated and uncoated nanofibers (Synthetic-ECM™, product number P609186, Donaldson Co., MN) and coated and uncoated polystyrene. At day 2, 5-bromodeoxyuridine (BrdU, 1 μM concentration, Sigma) was added to the cultures, which allowed for determination of the number of dividing cells. Cells were pulsed with BrdU for a period of 48 hours and then stained with an anti-BrdU antibody in order to perform immunocytochemistry. PC12 cells were permeabilized by the procedure of S.P Memberg & A.K. Hall ((1995) Neurobiol. 27:26-43). Cell cultures were incubated with the anti-BrdU antibody (1:100, Sigma) in blocking buffer (PBS, 0.5% Triton-X-100™, and 5% goat serum) for a period of one hour, rinsed with PBS and incubated with anti mouse IgG1 secondary antibody (1:200, Southern Biotech) in blocking buffer for an additional hour. Cultures were rinsed three times with PBS and the labeled cells were observed using an inverted microscope (Leica DMLA).

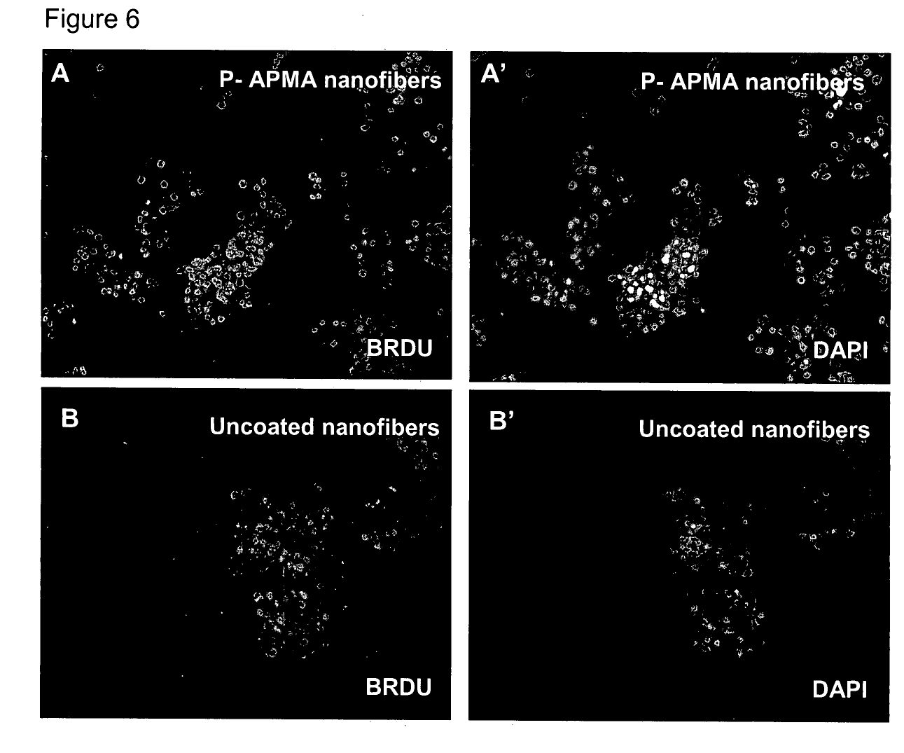

FIG. 6 shows a fluorescence microscopic image of BrdU incorporation in PC12 cells. The image indicates a greater incorporation of BrdU on photo-poly(APMA)-coated nanofibers as compared to uncoated nanofibers. Greater incorporation of BrdU on photo-poly(APMA)-coated surfaces compared to uncoated surfaces relates to a greater number of dividing cells, and is thought to be due to improved attachment of the cells on the photo-poly(APMA)-coated surfaces. The total number of cells present in a field is indicated by the DAPI staining. Cells grown on the photo-poly(APMA)-coated substrates showed 60% more incorporation of BrdU as compared to photo-polylysine-coated substrates.

### EXAMPLE 5

### Differentiation of PC12 Cells on Various Coated and Uncoated Surfaces

The differentiation of PC12 cells growing on photo-polymer-coated and uncoated polystyrene 12 well plates, photo-polymer-coated and uncoated nanofibers (Synthetic-ECM™, product number P609186, Donaldson Co., MN), PuraMatrix™ (BD Biosciences), and Matrigel™ (BD, Biosciences) was assessed in the presence of NGF (nerve growth factor).

Other photo reagents that were also tested included photo-fibronectin, photo-laminin, photo-collagen, photo-PEI and photo-RGD.

Cells were trypsinized and plated at a density of 30,000 cells/35 mm well in their normal growth media described in Example 3. Twenty four hours later, the growth media was replaced with the differentiation media (RPMI medium (Invitrogen) containing 1% horse serum, 0.5% fetal bovine serum, 2 mM Glutamax (Invitrogen), I mM sodium pyruvate (Invitrogen), and 1OmM HEPES (Invitrogen)). Cells were incubated at 37° C. in 5% CO₂/95% air humidified chamber and were differentiated for a period of 10 days with the addition of NGF (100 ng/mL, Invitrogen) every second day. To assess differentiation into neurons, the cells were stained for β-III tubulin after 10 days in culture. To perform immunostaining the cells were fixed with 4% paraformaldehyde for 20 min, washed with 0.1M PBS three times, and then incubated for an hour at room temperature with β-III tubulin primary antibody (1:200, Sigma), in PBS containing 0.5% Triton-X-100™ and 5% goat serum. Cells were washed and incubated with anti mouse secondary antibody (IgG2b, Southern Biotech) for an additional hour. Cells were washed and observed under an inverted microscope to visualize β-III tubulin staining.

Generally, the flat or three-dimensional surfaces that were coated with the photo-polymers produced differentiated cultures enriched in process bearing neurons. The average neurite length on these surfaces was doubled compared to uncoated surfaces. Other photo reagents (photo-laminin, photo-PEI and photo-collagen) were also found to be better than uncoated polystyrene or nanofibers if not better than photo-poly(APMA).

Hence, the photo-poly(APMA) coated surfaces produced better PC12 cell differentiation into neurons compared to uncoated polystyrene, uncoated nanofibers, collagen, PEI and commercially available PuraMatrixTm and Matrigel™ preparations.

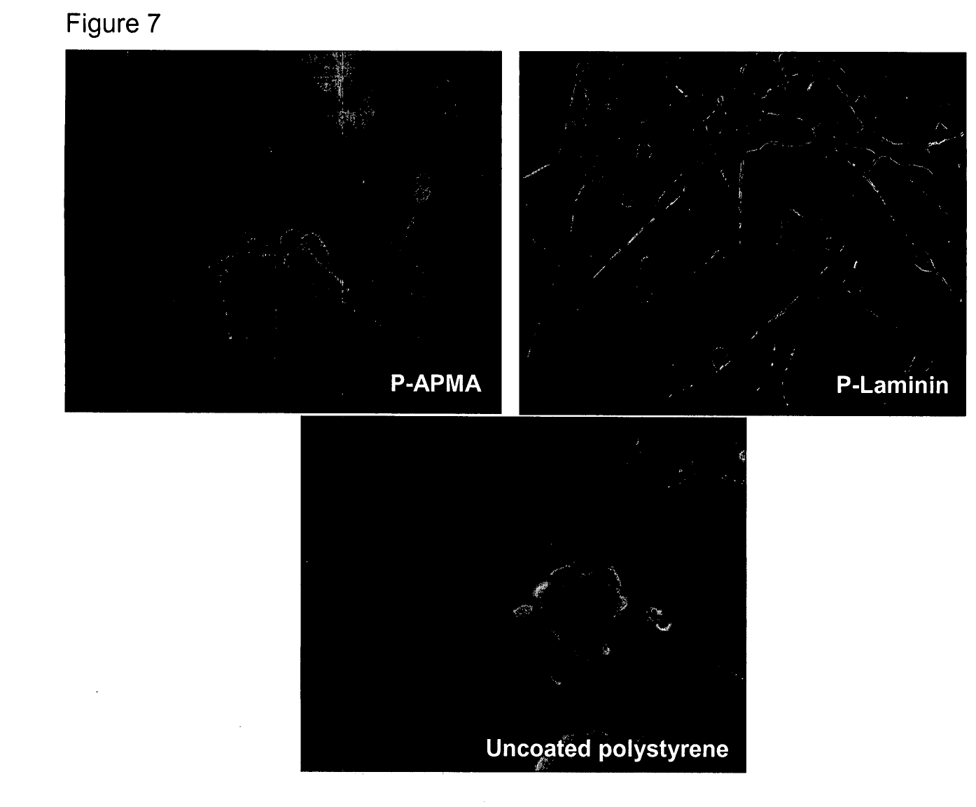

FIG. 7 shows that PC12 cells differentiate better on photo-poly(APMA) and photo-laminin coated polystyrene compared to uncoated polystyrene.

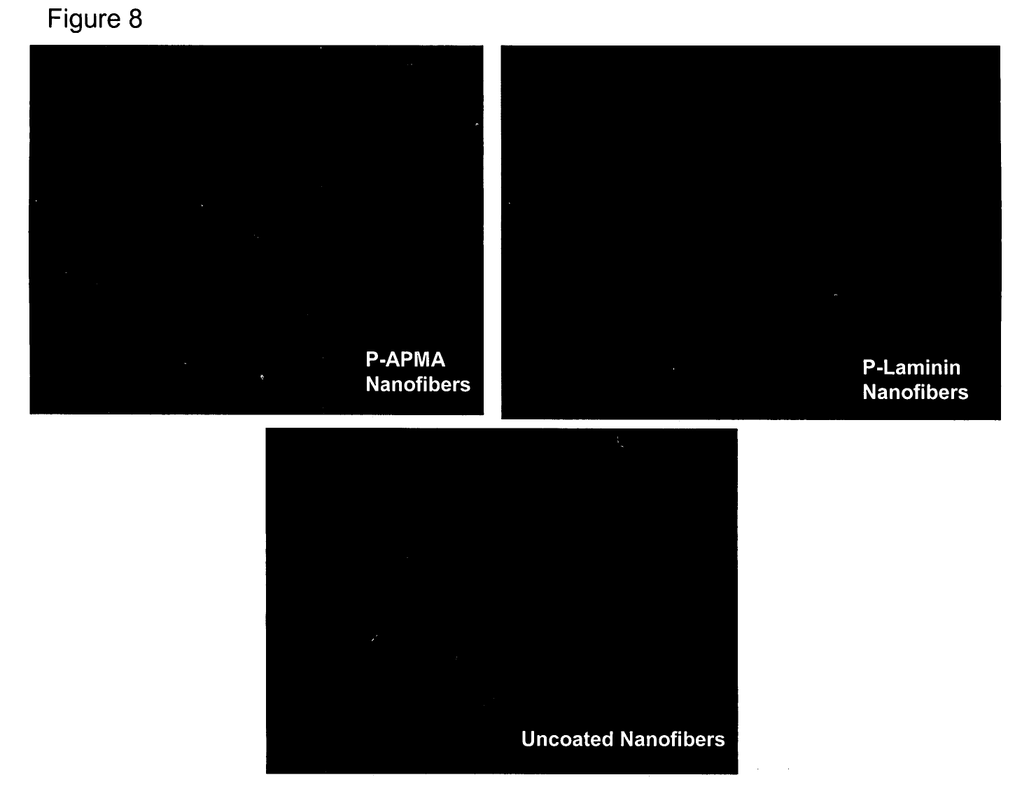

FIG. 8 shows that PC12 cells differentiate better on photo-poly(APMA) and photo-laminin coated nanofibers compared to uncoated nanofibers.

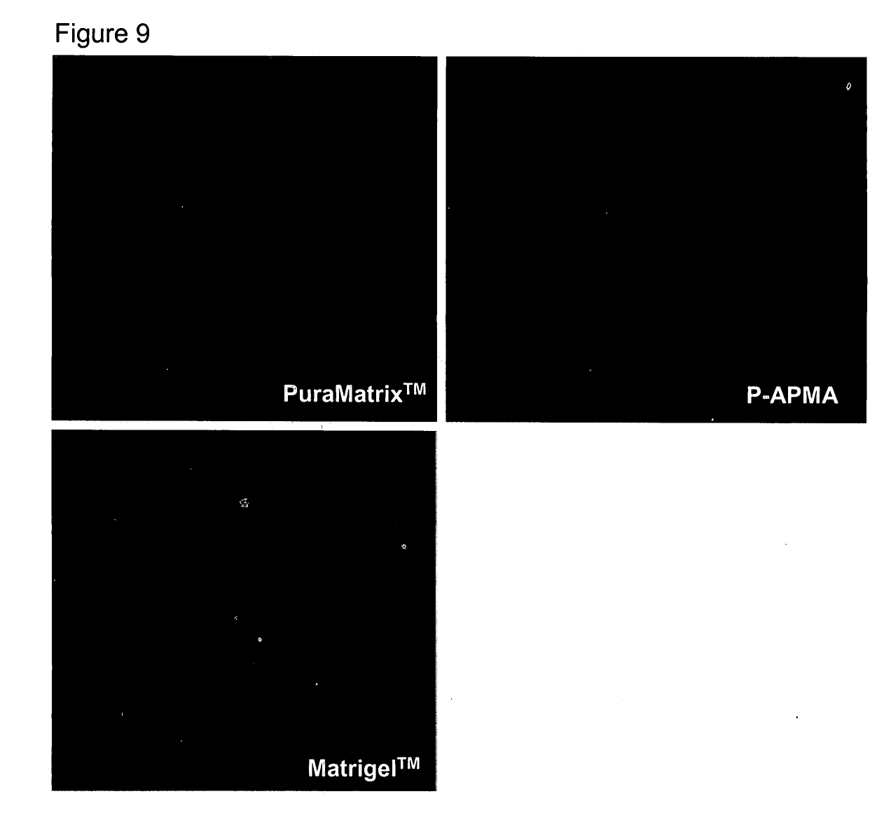

FIG. 9 shows that PC12 cells differentiate better on photo-poly(APMA) coated nanofibers compared to PuraMatrix™ and Matrigel™. Photo APMA promoted quantitatively better cell differentiation and longer neurite extension on nanofibers.

Assessments of β-III tubulin staining, neurite morphology, and cell differentiation are summarized in Table 2.

TABLE 2

| | | B-III | | |
|---|---|---|---|---|
| | | tubulin | Neurite | % |
| | | staining | morphology | Differentiation |
| Polystyrene | P-APMA | +++ | ++++ | 60% |
| 12-well plates | P-PEI | ++ | ++ | 20% |
| | P-FN | + | + | Less than 5% |
| | P-Collagen | +++ | +++ | 30% |
| | P-Laminin | ++++ | ++++ | 80% |
| | P-RGD | ++ | ++ | 10% |
| Uncoated polystyrene | | + | + | 1% |
| 12-well plates | | | | |
| Coated nanofibers | P-APMA | ++++ | ++++ | 50% |
| | P-PEI | ++ | ++ | 20% |
| | P-FN | + | + | Less than 5% |
| | P-Collagen | +++ | +++ | 30% |
| | P-Laminin | +++ | +++ | 30% |
| | P-RGD | ++ | ++ | 10% |

| | | | |
|---|---|---|---|
| Uncoated nanofibers | + | + | 1% |
| PuraMatrix ™ | +++ | +++ | 10% |
| Matrigel ™ | +++ | ++ | 5% |

++++ very good;
+++ good;
++ fair;
+ poor

### EXAMPLE 6

### Proliferation of ES-D3 Cells on Photo-Polymer Coated Surfaces

ES-D3 cells from ATCC (Acc. Number CRL-1934) were grown as aggregates in suspension dishes (Nunc) in DMEM-F12 (Invitrogen) with 10% fetal calf serum (FCS, Cambrex) and leukemia inhibitory factor (LIF, 10 ng/ml, Gibco-BRL) for 4 days. The medium was then changed to a chemically defined medium called NEP basal medium (DMEM-F-12 supplemented with 100 μg/ml transferrin, 5 μg/ml insulin, 16 μg/ml 10 putrescine, 20 nM progesterone, 30 nM selenious acid, 1 mg/ml bovine serum albumin, 20 ng/ml bFGF plus B27 and N2 additives and the cells were seeded on fibronectin (15 μg/ml, Sigma) coated polystyrene dishes, photo poly(APMA)-coated and uncoated nanofibers (Synthetic-ECM™, product number P610304, Donaldson Co., MN). Medium was changed every 2 days and the cells were maintained at 37° C. in 5% CO₂/95% air humidified chamber (Mujtaba and Rao (1999) Developmental Biology 214:113-127). Photo-poly(APMA)-coated surfaces were used with the ES-D3 cells Nestin Staining for proliferating ES-D3 cells

The ES-D3 cells were assayed at 24 hours and 48 hours. ES-D3 cells were stained for the presence of nestin, a marker for undifferentiated stem cells (U. Lendahl et al. (1990) Cell 60:585-95) as follows. Cells were fixed for 20 min at room temperature with 4% paraformaldehyde. They were washed three times with 0.1 M PBS, pH 7.4 and incubated with primary antibody to rat nestin (rat 401, DSHB) diluted 1:1 diluted in PBS containing 0.5% Triton-X-100™ and 5% goat serum for two hours at room temperature. Cells were then washed for 5 min with 0.1M PBS and incubated with anti mouse secondary (1:200, Southern Biotech) diluted in PBS containing 0.5% Triton-X-100™ and 5% goat serum for an additional hour after which they were washed three times with 0.1 M PBS and observed under an inverted microscope (Leica, DMLA). Nestin was double labeled with BrdU and the double labeling experiments were performed by simultaneously incubating cells in appropriate combinations of primary antibodies followed by non-cross reactive secondary antibodies.

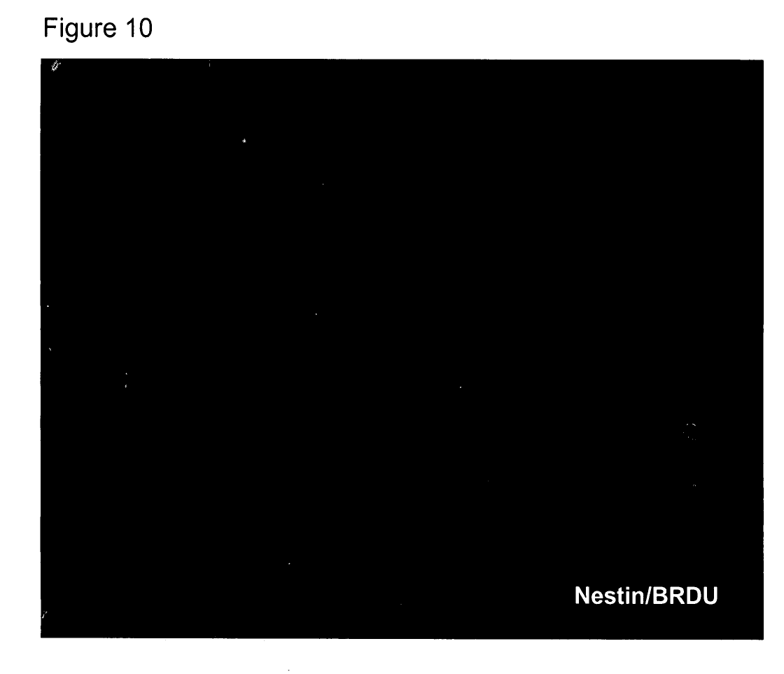

FIG. 10 shows the presence of nestin/BrdU positive ES-D3 cells on coated nanofibers. The majority of the cells are nestin positive and negative for all other lineage markers tested (GFAP, β-III tubulin, and O4) which indicates that cultures of undifferentiated stem cells can be maintained on these totally synthetic surfaces coated with photo-poly(APMA).

### EXAMPLE 7

### Differentiation of ES-D3 Cells on Coated Surfaces

Nestin positive ES-D3 cells growing on photo-poly(APMA) coated nanofibers (Synthetic-ECM™, product number P609192, Donaldson Co., MN) in basal medium (DMEM-F-12 supplemented with 100 μg/ml transferrin, 5 μg/ml insulin, 16 μg/ml putrescine, 20 nM progesterone, 30 nM selenious acid, 1 mg/ml bovine serum albumin, 20 ng/ml bFGF plus B27 and N2 additives). Cells were induced to differentiate by removal of bFGF from the growth medium and addition of either retinoic acid (1 μM, Sigma), PDGF BB (10 ng/ml, Sigma), CNTF (10 ng/ml, Sigma), 10% serum (FBS, Invitrogen) (Mujtaba and Rao, ibid.). After six days in culture with the daily addition of inducing agents the stem cells differentiated into neurons, oligodendrocytes and astrocytes. The differentiation was achieved without changing the substrate of the cells. β-III tubulin staining

The cells were fixed with 4% paraformaldehyde for 20 min, washed with 0.1 M PBS three times and incubated for an hour at room temperature with β-III tubulin (1:200, Sigma), a marker for neurons in PBS containing 0.5% Triton-X-100™ and 5% goat serum. Cells were washed and incubated with anti mouse secondary antibody (IgG2b, Southern Biotech) for an additional hour. Cells were washed and observed under an inverted microscope.

In some instances we also stained with DAPI as follows. Cells prepared as above were washed with DAPI solution (diluted 1:1000 in 100% MeOH, Boehringer Mannheim). Fixed cells were incubated with DAPI solution for 15 min at room temperature.

O4 Staining for Oligodendrocytes

Cells were fixed for 10 min at room temperature with 4% paraformaldehyde. Cells were washed three times for 5 min with 0.1 M PBS, pH 7.4. Cells were incubated with primary antibodies to O4 (6 μg/ml, Chemicon) in medium containing 5% BSA for two hours at room temperature. Preparations were then washed three times for 5 min with 0.1 M PBS, pH 7.4. Cells were incubated with secondary antibodies, and further processed as described above for P-III tubulin.

GFAP Staining for Astrocytes

Cells were fixed for 20 min at room temperature with 4% paraformaldehyde. Cells were washed three times with 0.1 M PBS, pH 7.4. Cells were incubated with primary antibody to GFAP (1:500, Chemicon) in PBS containing 0.5% Triton-X-100™ and 5% goat serum for two hours at room temperature. Cells were washed and incubated with anti mouse secondary antibody and further processed as described above for β-III tubulin.

Uncoated nanofibers and polystyrene, double coated with poly-lysine (15 ug/ml, Sigma) and laminin (15 ug/ml, Gibco BRL), were used as controls.

Better differentiation into appropriate cell types is achieved on coated nanofibers compared to uncoated nanofibers and tissue culture plastic double coated with poly-lysine/laminin, and that this differentiation is achieved by the mere addition of appropriate inducing agents to the same substrate.

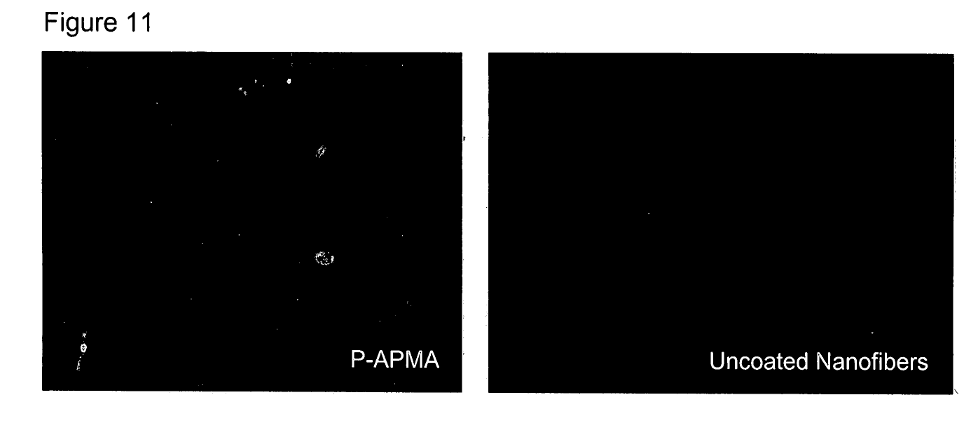

FIG. 11 shows that ES-D3 cells differentiate into process bearing neurons with longer neurite lengths compared to neurons growing on uncoated nanofibers where the processes are short and stubby.

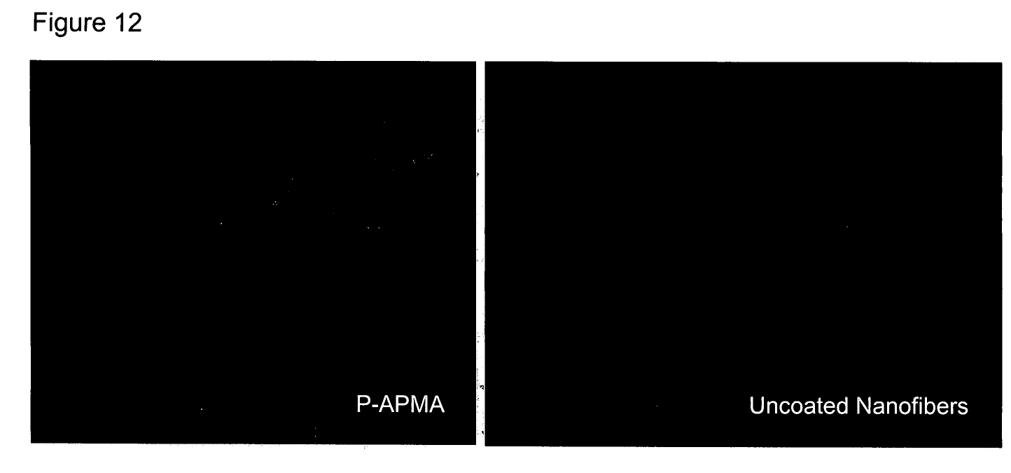

FIG. 12 shows that ES-D3 cells differentiate into Type I and Type II GFAP positive astrocytes on photo-poly(APMA) coated nanofibers. Note that the cells are much brighter on coated nanofibers.

### EXAMPLE 8

### Growth of PC12 Cells at Clonal Density

PC12 cells were plated at a density of 300 cells/32 mm nanofiber disc (Synthetic-ECM™, product number P609186, Donaldson Co., MN) placed in a 35 mm well and were differentiated for a period of 30 days with the addition of NGF every second day (as described in Example 5). After 30 days, the cells were fixed with 4% paraformaldehyde for 20 min, washed with 0.1 M PBS three times and incubated for an hour at room temperature with β-III tubulin (1:200, Sigma) in PBS containing 0.5% Triton-X-100™ and 5% goat serum. Cells were washed and incubated with anti mouse secondary antibody (IgG2b, Southern Biotech) for an additional hour. Cells were washed and observed under an inverted microscope. Single cells started to form clones after four days in culture. The clones grew rapidly and remained firmly attached to the coated surfaces.

### EXAMPLE 9

### Long Term Cultures of PC12 Cells on Photo-Polymer Coatings

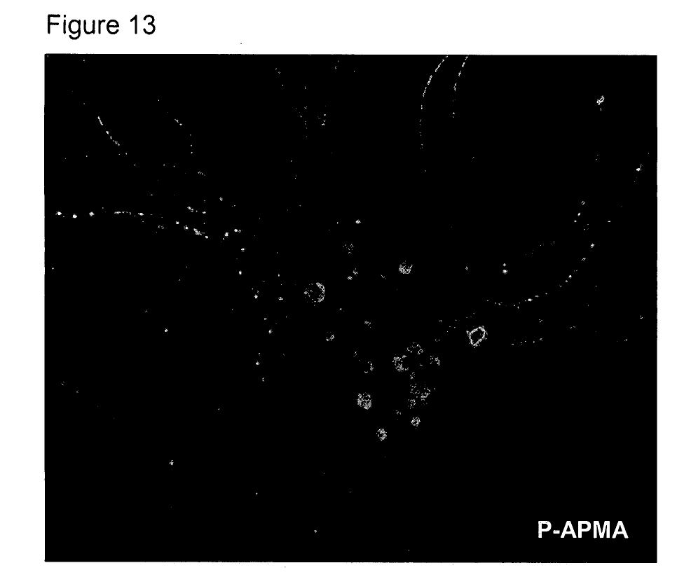

PC12 cells cultured as described in Example 8 were maintained for a period of 45 days and processed for β-III tubulin. FIG. 13 shows differentiated PC12 cells growing on photo-poly(APMA)-coated surfaces. After 30 days in culture, the cells were robust and displayed extensive neurites.

### EXAMPLE 10

### Long Term Cultures of ES-D3 Cells on Photo-Polymer Coatings

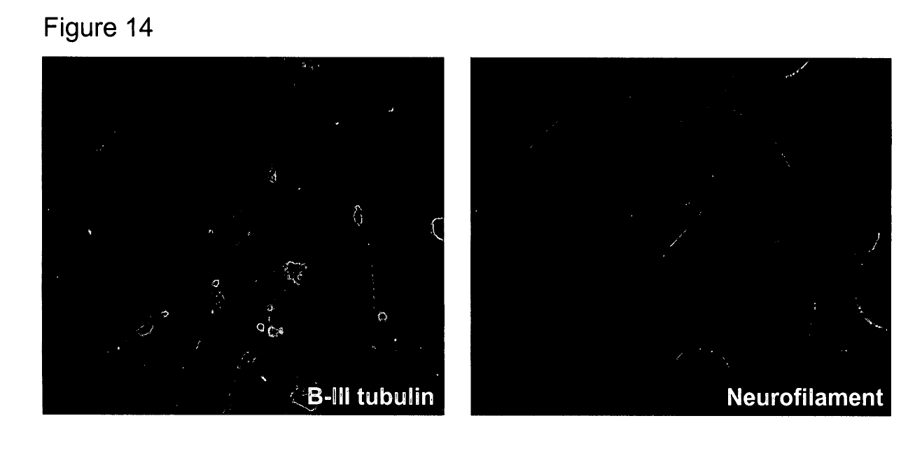

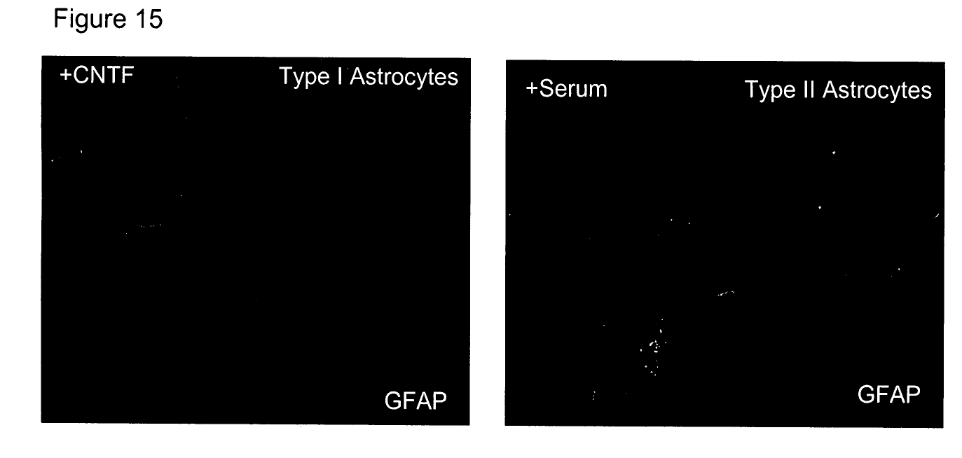

ES-D3 cells cultured and differentiated as described in Example 7 were maintained over a period of 22 days and processed for β-III tubulin and Neurofilament and GFAP. FIG. 14, shows the maturation of stem cells into β-III tubulin positive neurons. A subset of these neurons is also Neurofilament⁺. At this time point in culture the cells growing on bare nanofibers and poly-Lysine/Laminin started to detach from the substrates while the cells growing on APMA coated nanofibers remained firmly attached to the substrate and differentiated into process bearing mature neurons. FIG. 15, shows the maturation of astrocytes into GFAP⁺ Type I and Type II astrocytes. The cells remain firmly attached to the surface while the cells growing on bare nanofibers and poly-Lysine/Laminin started to come off the substrates.

What is claimed is:

1. A cell culture article comprising:
(a) a nanofibrillar structure comprising nanofibers, and
(b) a coating formed on at least a portion of the nanofibers, the coating comprising a polymer comprising (i) at least one pendent amine-containing group and (ii) at least one pendent latent reacted group that bonds the polymer to the nanofibers.

2. The cell culture article of claim 1 wherein the polymer is a non-biodegradable polymer.

3. The cell culture article of claim 1 wherein the polymer is a synthetic polymer.

4. The cell culture article of claim 3 wherein the pendent amine-containing group has the formula: —R₁R₂NR₃R₄
wherein R₁ is: 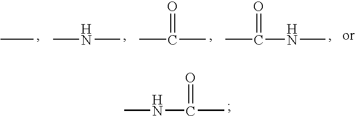
wherein R₂ is C₁-C₈ linear or branched alkyl; and
wherein R₃ and R₄ are both attached to the nitrogen and are individually H or C₁-C₆ linear or branched alkyl.

5. The cell culture article of claim 4 wherein the pendent amine-containing group has the formula: —R₁R₂NR₃R₄ and
R₁ is 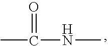
R₂ is C₂-C₄ linear or branched alkyl; and R₃ and R₄ are both attached to the nitrogen and are individually H, CH₃, or C₂H₅

6. The cell culture article of claim 5 wherein the polymer comprises one or more monomers selected from the group consisting of 3-aminopropylmethacrylamide, 3-aminoethylmethacrylamide, and dimethylaminopropylmethacrylamide.

7. The cell culture article of claim 6 wherein the polymer comprises 3-aminopropylmethacrylamide (APMA).

8. The cell culture article of claim 4 wherein the pendent amine-containing group is present in a molar amount of 50% or greater of the polymer based on total monomer units in the polymer.

9. The cell culture article of claim 1 wherein the pendent latent reacted group comprises a photoreacted group.

10. The cell culture article of claim 9 wherein the photoreacted group is selected from the group consisting of acetophenone, benzophenone, anthraquinone, anthrone, and anthrone-like heterocycles, and substituted derivatives thereof.

11. The cell culture article of claim 10 wherein the photoreacted group is present in a molar amount in the range of 0.1% to 10% based on total monomer units in the polymer.

12. The cell culture article of claim 3 wherein the synthetic polymer has a molecular weight in the range of 20 kDa to 2000 kDa.

13. The cell culture article of claim 1 wherein the nanofibers comprise a non-biodegradable polymer.

14. The cell culture article of claim 13 wherein the nanofibers comprise a water-soluble or short chain alcohol-soluble polymer.

15. The cell culture article of claim 14 wherein the nanofibers comprise a crosslinking agent.

16. The cell culture article of claim 15 wherein the nanofibers are formed by a method that includes a step of activating the crosslinking agent by UV treatment, heat, or a combination thereof.

17. The cell culture article of claim 13 wherein the nanofibers comprise a polyamide.

18. The cell culture article of claim 17 wherein the polyamide is selected from the group consisting of nylon polymers.

19. The cell culture article of claim 18 wherein the nanofibers comprise a blend of two or more nylon polymers.

20. The cell culture article of claim 1 wherein the nanofibers have diameters in the range of 50 nm to 1000 nm.

21. The cell culture article of claim 1 wherein the nanofibrillar structure comprises a network of the nanofibers, said network having an average pore size in the range of about 0.2 μm to 10 μm.

22. The cell culture article of claim 1 wherein the nanofibrillar structure has a thickness in the range of 0.1 μm to 10 μm.

23. The cell culture article of claim 1 wherein the nanofibrillar structure has an area in the range of 1 mm² to 4×10⁵ mm².

24. The cell culture article of claim 1 further comprising a support for the nanofibrillar structure.

25. The cell culture article of claim 24 wherein the support comprises a halogenated thermoplastic resin.

26. The cell culture article of claim 25 wherein the support comprises chlorotrifluoroethylene.

27. The cell culture article of claim 24 wherein the support comprises glass.

28. The cell culture article of claim 24 wherein the support is transparent.

29. The cell culture article of claim 24 wherein the support is flexible.

30. The cell culture article of claim 24 wherein the nanofibrillar structure is formed by disposing nanofibers on the support and then treating the disposed nanofibers and support with heat, steam, or a combination thereof.

31. The cell culture article of claim 24 adapted for placement onto a cell culture surface of a cell culture vessel.

32. The cell culture article of claim 31 wherein the cell culture vessel is selected from the group consisting of a flask, a dish, and a multi-well plate.

33. The cell culture article of claim 1, further comprising cells in contact with said substrate.

34. A method for preparing a cell culture article comprising the steps of:
(a) obtaining a nanofibrillar structure comprising nanofibers, wherein a coating is formed on at least a portion of the nanofibers, the coating comprising a polymer having (i) at least one pendent amine-containing group and (ii) at least one pendent latent reacted group that bonds the polymer to the nanofibers; and
(b) providing the nanofibrillar structure onto a cell culture surface of a cell culture vessel.

35. A method for preparing a cell culture article comprising the steps of:
(a) obtaining a nanofibrillar structure comprising nanofibers;
(b) disposing a coating composition on at least a portion of the nanofibers, the coating composition comprising a polymer having (i) at least one pendent amine-containing group and (ii) at least one pendent latent reactive group; and
(c) treating the coating composition to activate the pendent latent reactive group and bond the polymer to the nanofibers via a latent reacted group, thereby forming a coating on the nanofibers.

36. The method of claim 35 wherein the polymer is present in the coating composition in the range of 10 μg/mL to 20 mg/mL.

37. The method of claim 36 wherein the latent reactive group is a photoreactive group.

38. The method of claim 35 wherein the step of treating comprises irradiating the photoreactive group with UV light in the range of 190 nm to 360 nm.

39. The method of claim 38 wherein the step of treating comprises irradiating the photoreactive group with a dose of UV light in the range of 0.1 mW/cm² to 20 mW/cm².

40. A method for culturing one or more cells comprising placing the one or more cells in contact with a nanofibrillar structure comprising nanofibers, wherein the nanofibers comprise a coating formed on at least a portion of the nanofibers, the coating comprising a polymer having (i) at least one pendent amine-containing group and (ii) at least one pendent latent reacted group that bonds the polymer to the nanofibers.

41. The method of claim 40 wherein 90% of the cells are attached to the coated nanofibers.

42. The method of claim 40, wherein the cells are placed in contact with the nanofibrillar structure for a period of 14 days or more.

43. The method of claim 40 wherein the cells are selected from the group consisting of mammalian, avian, piscine, reptilian, amphibian, and insect cells.

44. The method of claim 40 wherein the cells are somatic cells.

45. The method of claim 44 wherein the somatic cells are stem cells.

46. The method of claim 45 wherein the stem cells are maintained in an undifferentiated state for a period of time between one hour and 60 days.

47. The method of claim 46 wherein the viability of the undifferentiated cells is not reduced by more than 90% at 14 days.

48. The method of claim 40 further comprising a step of inducing the stem cells to differentiate.

49. The method of claim 40 wherein the cells are neuronal precursor cells.

50. The method of claim 49 wherein neuronal precursor cells are placed in contact with the nanofibrillar structure under conditions and for a period of time sufficient for the neuronal precursor cells to develop into neurons.

51. The method of claim 49 wherein the neuronal precursor cells are placed in contact with the nanofibrillar structure under conditions and for a period of time sufficient for the neuronal precursor cells to develop into oligodendrocytes.

52. The method of claim 49 wherein the neuronal precursor cells are placed in contact with the nanofibrillar structure under conditions and for a period of time sufficient for the neuronal precursor cells to develop into astrocytes

53. The method of claim 49 wherein greater than 30% of the neuronal precursor cells are differentiated after a period of 10 days.

54. A method of producing oligodendrocytes, astrocytes, or neurons, comprising placing a cell selected from the group consisting of precursors of oligodendrocytes, astrocytes, and neurons in contact with a nanofibrillar structure comprising a coating formed on at least a portion of the nanofibrillar structure, the coating comprising a polymer having (i) at least one pendent amine-containing group and (ii) at least one pendent latent reacted group that bonds the polymer to the substrate, wherein the precursors are placed under conditions and for a period of time sufficient for the precursors to develop into the respective oligodendrocytes, astrocytes, or neurons.
